(19) United States
(12) Patent Application Publication
(10) Pub. No.: US 2024/0076274 A1
(43) Pub. Date: Mar. 7, 2024

(54) TRIAZINE-BASED SELF-ASSEMBLING SYSTEM

(71) Applicant: Council of Scientific and Industrial research, New Delhi (IN)

(72) Inventors: Chhuttan Lal MEENA, Pune (IN); Dharmendra SINGH, Pune (IN); Gangadhar Jessy SANJAYAN, Pune (IN)

(21) Appl. No.: 18/263,082

(22) PCT Filed: Jan. 25, 2022
(86) PCT No.: PCT/IN2022/050057

§ 371 Date: Jul. 26, 2023

(30) Foreign Application Priority Data
Jan. 26, 2021 (IN) 202111003499

(51) Int. Cl. C07D 251/46; C08F 116/14; C08F 120/34; C08F 120/60

(52) U.S. Cl. CPC C07D 251/46; C08F 116/14; C08F 120/34; C08F 120/60

(57) ABSTRACT

A Janus G-C base as building block for a triazine based self-assembly of formula (I), a process for the preparation, and its application in developing supramolecular polymers, peptide nucleic acids (PNAs) and smart polymers thereof. A triazine based self-assembly of formula (I):

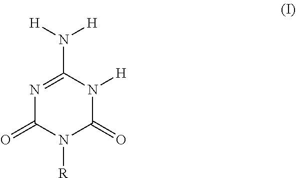

wherein, ‘R’ is selected from the group comprising of linear or branched unsubstituted and substituted C1-C7 alkyl, unsubstituted and substituted aryl, unsubstituted and substituted natural amino acids which may be protected, linear or branched unsubstituted and substituted C1-C7 alcohols, or linear or branched unsubstituted and substituted C1-C7 amines.

### FIELD OF THE INVENTION

The present invention relates to a triazine based self-assembling system. More particularly, the present invention relates to a Janus G-C base as building block for a triazine based self-assembly of formula (I), a process for the preparation, and its application in developing supramolecular polymers, peptide nucleic acids (PNAs) and smart polymers thereof.

### BACKGROUND OF THE INVENTION

Molecular self-assembling systems has ushered as a promising approach to construct complex nanostructures with wide range of attractive mechanical characteristics that find diverse applications such as in chemical synthesis, polymer science, nanotechnology, tissue engineering, in medical applications and for fabricating advanced materials.

By virtue of their structural similarity with nucleobases, synthetic molecular self-assembling systems have also found application in developing novel peptide nucleic acids (PNA)—a class of nucleic acid mimics made of pseudopeptide backbone with excellent recognition-specificity. Inspired by the nucleic acid base-pairing, researchers and chemists have pursued in developing such self-assembling molecules with diverse H-bonding characteristics as a building block for supramolecular polymer networks.

One of the extensively studied self-assembling motifs that instantaneously forms supramolecular polymers is the melamine-cyanuric/barbituric acid complementary dual motif (MA-CA/BA) is shown below:

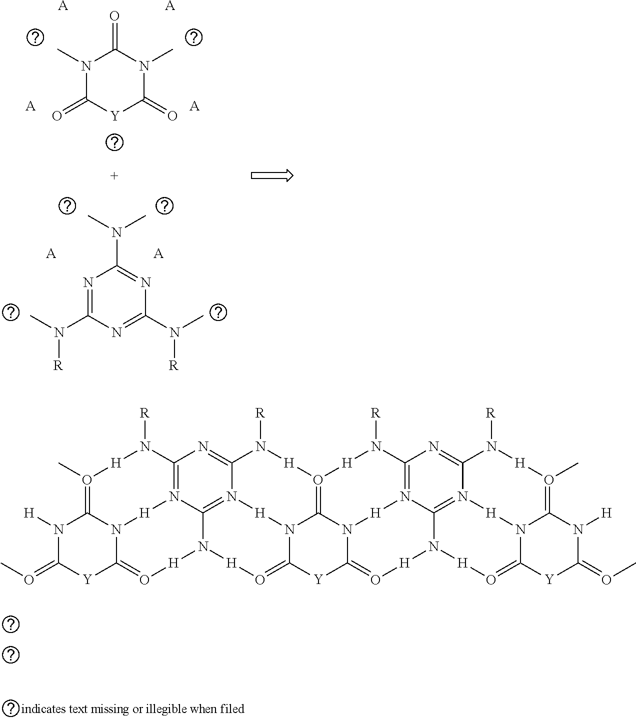

Depending upon the substitution pattern on melamine, MA-CA/BA assembly can give rise to three different types of aggregates: the cyclic rosettes (finite), linear tapes (infinite), and crinkled tapes (infinite), however, the multiple possibilities of self-assembly pathways are detrimental to applications particularly when formation of a mixture of polymeric aggregates is not desired. Further, the need to maintain equimolar proportion of the complementary self-assembling components for effecting self-assembly adds to the practical difficulties.

A need exists in the art for self-assembling motifs capable of instant supramolecular polymerization, but devoid of multiple assembly pathways that could be of considerable advantage for practical applications.

### OBJECTS OF THE INVENTION

The main object of the present invention is to provide a triazine based self-assembling Janus G-C-base nucleic acid motif of formula (I) that could possess advantageous applications. Another object of the present invention is to provide a process for the synthesis of a triazine based self-assembling Janus G-C-base nucleic acid motif of formula (I).

Yet another object of the present invention is to provide an application of a triazine based self-assembling Janus G-C-base nucleic acid motif of formula (I) in developing supramolecular polymers, peptide nucleic acids (PNAs) and smart polymers.

### SUMMARY OF THE INVENTION

The primary objective of the present invention is to provide a triazine based self-assembling Janus G-C-base motif of formula (I) capable of efficient self-assembly, leading exclusively to a single set of linear supramolecular polymer thereby avoiding formation of mixture of supramolecular polymers.

In an aspect of an embodiment, the present invention provides a triazine based self-assembling Janus G-C-base motif of formula (I):

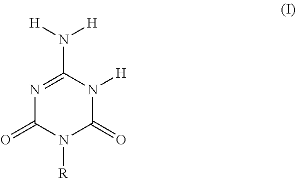

wherein, ‘R’ is selected from the group comprising of linear or branched unsubstituted and substituted C1-C7 alkyl, unsubstituted and substituted aryl, unsubstituted and substituted natural amino acids which may be protected, linear or branched unsubstituted and substituted C1-C7 alcohols, or linear or branched unsubstituted and substituted C1-C7 amines. In another embodiment, the present invention provides a process for the synthesis of a triazine based self-assembling Janus G-C-base motif of formula (I); wherein the process comprises the steps of:
i. reacting guanidine (1) with BOC in presence of base, suitable solvent and water at a temperature in the range of 25-30° C. for a period in the range of 9-10 hours to obtain Boc-protected guanidine (2):
ii. reacting the compound (2) obtained at step i) with CDI in a suitable solvent at a temperature in the range of 25-30° C. for a period in the range of 5-6 hours to obtain the imidazole carbonyl-coupled reactive intermediate (3);
iii. reacting the intermediate (3) obtained at step ii) with R—NH₂ (II) in the presence of a base and a suitable solvent at a temperature in the range of 45-50° C. for a period in the range of 4-5 hours to obtain amidino urea (4):
iv. cyclizing the amidino urea (4) obtained at step iii) with CDI at a temperature in the range of 30-35° C.for a period in the range of 45-48 hours to obtain the Boc protected Janus G-C nucleobase (5); and
v. deprotecting the nucleobase (5) obtained at step iv) at a temperature in the range of 0-4° C. for a period in the range of 40-45 minutes to obtain the compound of formula (I).

In an aspect of an embodiment, the present invention provides an intermediate (3) possessing below structural formula:

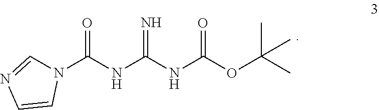 [SMILES: CC(C)(C)OC(=O)NC(=N)NC(=O)n1ccnc1]

Yet another aspect of an embodiment, the present invention provides a process for the preparation of an intermediate (3), wherein said process comprises the steps of:
a) reacting guanidine (1) with BOC in presence of base, suitable solvent and water at a temperature in the range of 25-30° C. for a period in the range of 9-10 hours to obtain Boc-protected guanidine (2);
b) reacting the compound (2) obtained at step i) with CDI in equimolar amount in a suitable solvent at a temperature in the range of 25-30° C. for a period in the range of 5-6 hours to obtain the intermediate (3).

Yet another embodiment of the present invention provides an application of a triazine based self-assembling Janus G-C-base motif of formula (I) in developing a supramolecular polymer, peptide nucleic acids (PNAs) and smart polymers.

### DETAILED DESCRIPTION OF THE DRAWINGS

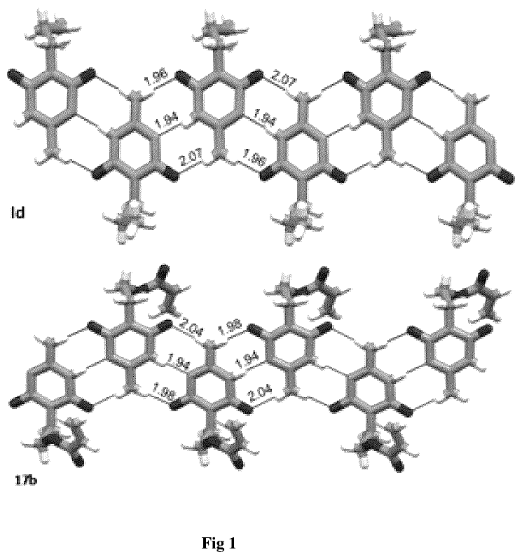

FIG. 1: Depicts Single-crystal X-ray structures of triazine-based Janus G-C nucleobases 6d and 17b showing supramolecular self-assembly. H-bonding is highlighted in dashes, above which hydrogen bond distances (N—H . . . N, N . . . H—N and N—H . . . O) are displayed in Å.

### ABBREVIATIONS

t-Boc—tert-butyloxycarbonyl
Pbf—2,2,4,6,7-pentamethyldihydrobenzofuran-residue
CDI—carbonyldiimidazole
Bn—benzyl
Cbz—carboxybenzyl or benzyloxycarbonyl
Fmoc—Fluorenylmethyloxycarbonyl
HBTU—2-(1H-benzotriazol-1-yl)-1,1,3,3-tetramethyluronium hexafluorophosphate
HOBt—Hydroxybenzotriazole
Cbz-OSu—Benzyl N-succinimidyl carbonate
PTSA—p-toluene sulphonic acid

### DETAILED DESCRIPTION OF THE INVENTION

The invention will now be described in detail in connection with certain preferred and optional embodiments, so that various aspects thereof may be more fully understood and appreciated.

The present invention has developed a novel class of bifacial triple hydrogen-bonding a triazine based self-assembling Janus G-C-base motif of formula (I), inspired by DNA base-pairing. These nucleobases could serve as potential building blocks for multiple application purposes vis-a-vis: molecular self-assembly, nucleic acid interactions and smart polymers. The Janus G-C base, endowed with self-complementary H-bonding codes reminiscent of guanine (G) and cytosine (C) nucleic acid bases, is capable of undergoing efficient self-assembly leading to supramolecular polymers, peptide nucleic acids (PNAs) and smart polymers.

In an embodiment, the present invention provides a triazine based self-assembling Janus G-C-base motif of formula (I):

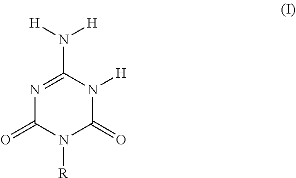

wherein, ‘R’ is selected from the group comprising of linear or branched unsubstituted and substituted C1-C7 alkyl, unsubstituted and substituted aryl, unsubstituted and substituted natural amino acids which may be protected, linear or branched unsubstituted and substituted C1-C7 alcohols, or linear or branched unsubstituted and substituted C1-C7 amines.

In a preferred embodiment, a triazine based self-assembling Janus G-C-base motif of formula (I) comprises of:
6-amino-3-benzyl-1,3,5-triazine-2,4 (1H,3H)-dione (Ia); 6-amino-3-neopentyl-1,3,5-triazine-2,4 (1H,3H)-dione (Ib); 6-amino-3-(2-hydroxyethyl)-1,3,5-triazine-2,4(1H,3H)-dione (Ic); 6-amino-3-(2-aminoethyl)-1,3,5-triazine-2,4(1H,3H)-dione (Id); Benzyl 2-(4-amino-2,6-dioxo-3,6-dihydro-1,3,5-triazin-1(2H)-yl) acetate (Ie); Benzyl 2-(4-amino-2,6-dioxo-3,6-dihydro-1,3,5-triazin-1(2H)-yl)-6-(((benzyloxy) carbonyl) amino) hexanoate (If); and Benzyl (S)-2-(4-amino-2,6-dioxo-3,6-dihydro-1,3,5-triazin-1(2H)-yl)-5-guanidino pentanoate (Ig).

In another embodiment, the present invention provides a process for the synthesis of a triazine based self-assembling Janus G-C-base motif of formula (I); wherein the process comprises the steps of:
i. reacting guanidine (1) with BOC in presence of base, suitable solvent and water at a temperature in the range of 25-30° C. for a period in the range of 9-10 hours to obtain Boc-protected guanidine (2);
ii. reacting the compound (2) obtained at step i) with CDI in a suitable solvent at a temperature in the range of 25-30° C. for a period in the range of 5-6 hours to obtain the imidazole carbonyl-coupled reactive intermediate (3);
iii. reacting the intermediate (3) obtained at step ii) with R—NH₂ (II) in the presence of a base and a suitable solvent at a temperature in the range of 45-50° C. for a period in the range of 4-5 hours to obtain amidino urea (4);
iv. cyclizing the amidino urea (4) obtained at step iii) with CDI at a temperature in the range of 30-35° C. for a period in the range of 45-48 hours to obtain the Boc protected Janus G-C nucleobase (5); and
v. deprotecting the nucleobase (5) obtained at step iv) at a temperature in the range of 0-4° C. for a period in the range of 40-45 minutes to obtain the compound of formula (I).
   wherein ‘R’ in compounds I and II are selected from the group comprising of linear or branched unsubstituted and substituted C1-C7 alkyl, unsubstituted and substituted aryl, unsubstituted and substituted natural amino acids which may be protected, linear or branched unsubstituted and substituted C1-C7 alcohols, or linear or branched unsubstituted and substituted C1-C7 amines.

In the present process, the solvent for the reaction is selected from polar or non-polar, protic or aprotic solvent such as lower alcohols, nitriles, ketones, halogenated hydrocarbons, TFA or combinations thereof. In a particularly useful embodiment, solvent is acetonitrile.

The base for the reaction is selected from organic bases such as ethylamine, triethylamine, DIPEA, pyridine or from inorganic base such as sodium hydroxide, alkali or alkaline earth metal carbonates and bicarbonates or combination thereof. In a particularly useful embodiment, base is DIPEA.

The deprotecting agent in step (v) of the present invention depends upon the protecting agent of the nucleobase (5). Accordingly, the debenzylation is carried out using H2/Pd-C, the Boc protecting group and the Pbf protecting groups are deprotected using 95% TFA in DCM.

The process is depicted in Scheme-1 below:

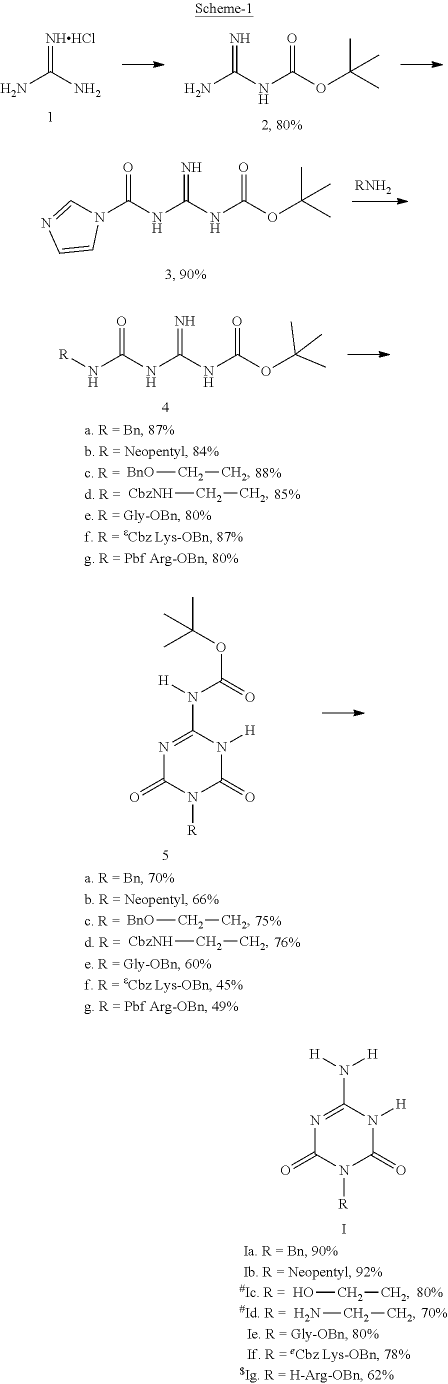

Another aspect of an embodiment provides an intermediate, N-tert-Butoxycarbonylguanidin-1H-imidazole-1-carbonyl, of formula (3);

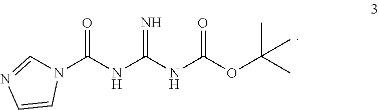 [SMILES: CC(C)(C)OC(=O)NC(=N)NC(=O)n1ccnc1]

Yet another aspect of an embodiment provides a process for preparation of the intermediate (3), wherein said process comprises the steps of:
a) reacting guanidine (1) with BOC in presence of base, suitable solvent and water at a temperature in the range of 25-30° C. for a period in the range of 9-10 hours to obtain Boc-protected guanidine (2);
b) reacting the compound (2) obtained at step i) with CDI in a suitable solvent at a temperature in the range of 25-30° C. for a period in the range of 5-6 hours to obtain the imidazole carbonyl-coupled reactive intermediate (3).

PNA monomer building blocks carrying unnatural nucleobases have found application in developing PNAs for site-specific interaction with DNAs and RNAs. PNA oligomers have been shown to inhibit transcription (antigene) and translation (antisense) of genes by tight binding to DNA or mRNA.

In another embodiment, the present invention provides a process for the synthesis of a triazine-based Janus G-C nucleobase containing PNA building blocks of formula 13 and 14, wherein said process comprises the steps of:
I. converting Cbz-ethylenediamine (7) to Cbz-protected ethyl (2-aminoethyl) glycinate (Cbz-AEG-OEt) (8); in presence of suitable solvent and base at a temperature in the range of 20-25° C. for a period in the range of 5-6 hours;
II. N-Boc ethylene diamine (9) reacted with benzyl bromoacetate in presence of suitable solvent and base at a temperature in the range of 30-35° C. for a period in the range of 5-6 hours to obtain Boc-protected benzyl ester (10);
III. Boc-protected benzyl ester (10) which was Boc deprotected in TFA-DCM (1:1) at 0-4° C. for 40-45 minute, was further reacted with Fmoc-OSu in presence of suitable base and solvent at a temperature in the range of 30-35° C. for a period in the range of 1-2 h hours to obtain Fmoc-protected benzyl-2 glycinate (Fmoc-AEG-OBn) (11).
IV. debenzylating glycine derived (5e) and/or arginine derived (5g) Boc-protected triazine benzyl esters obtained in Scheme 1 to carboxylic acid (12a, b) by stirring at a temperature in the range of 30-35° C. under hydrogenation with H₂/Pd-C for a period in the range of 5-6 hours;

V. coupling the intermediate (8) or (11) of step (I) and (III) at a temperature in the range of 30-35° C. for a period in the range of 12-16 hours with the compound (12a, b) to obtain the Fmoc analog (13a, b) and Cbz analog (14a, b), respectively.

The reaction is depicted in Scheme 2 below:

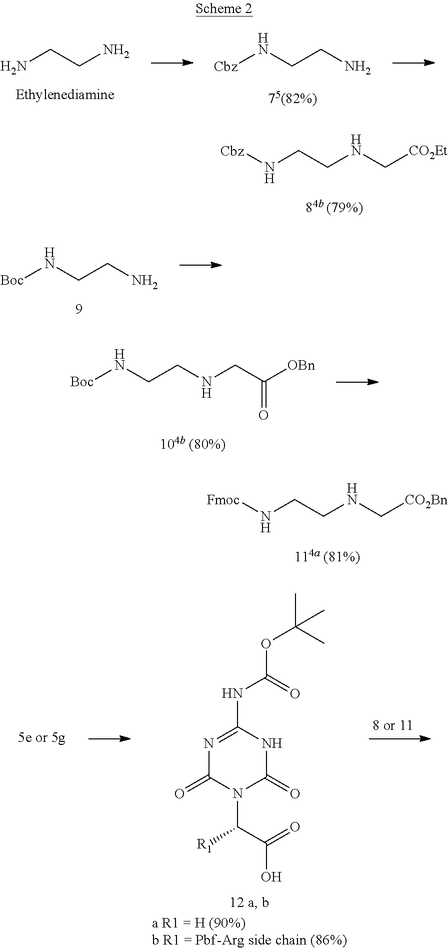

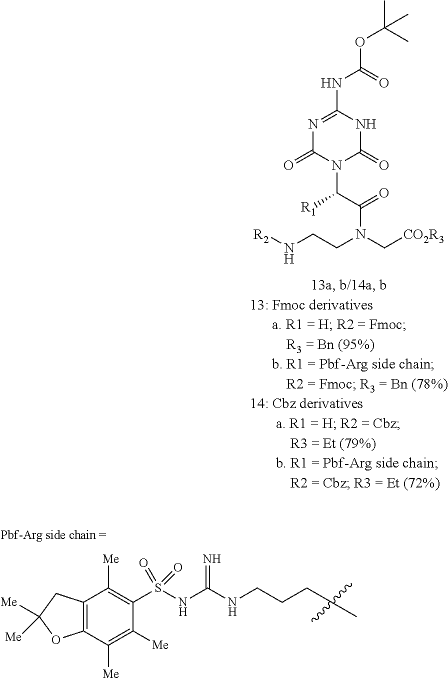

According to the process, ethylenediamine (7) is reacted with Cbz-OSu in a solvent-water mixture in 1:1 ratio to obtain Cbz protected ethylene diamine which is further reacted with ethyl bromoacetate in presence of base and solvent to yield Cbz-protected ethyl (2-aminoethyl) glycinate (Cbz-AEG-OEt) (8).

In another pot, N-Boc ethylene diamine (9) is reacted with benzyl bromoacetate in presence of solvent and base to yield the Boc-protected ester (10) which is Boc deprotection in TFA-DCM (1:1) and further reacted with Fmoc-OSu in presence of base and solvent to obtain Fmoc-protected benzyl 2 glycinate (Fmoc-AEG-OBn) (11).

The glycine derived (5e) and/or arginine derived (5g) Boc-protected triazine benzyl esters obtained in Scheme 1 are debenzylated by hydrogenation with H₂/Pd-C to yield the carboxylic acid (12a, b).

One part of the compound (12a, b) is dissolved in dry solvent and added HBTU, HOBt in presence of base and stirred, followed by addition of compound (11) to afford the Fmoc analog (13a, b).

The remaining part of the compound (12a, b) is dissolved in dry solvent and added HBTU, HOBt in presence of base and stirred, followed by addition of compound (8) to afford the Cbz analog (14a, b).

The solvent for the process is selected from polar or non-polar, protic or aprotic solvent such as lower alcohols, nitriles, ketones, halogenated hydrocarbons, TFA or combinations thereof. The base for the reaction is selected from organic base such as ethylamine, triethylamine, DIPEA, pyridine or from inorganic base such as sodium hydroxide, alkali or alkaline earth metal carbonates and bicarbonates or combination thereof.

In an embodiment, the synthesis of a triazine-based Janus G-C nucleobase-containing PNA building blocks (13a, b) or (14a, b) are useful for Fmoc-based solid-phase synthesis or Cbz-based solution-phase synthesis of PNAs.

Hydrogen-bonding plays a vital role in the designing of smart polymers and functional materials featuring controlled self-assembly.

In another embodiment, the present invention relates to a process for the synthesis of polymer building blocks 16 containing the Janus G-C nucleobase unit in a protected form, wherein said process comprises the steps of:
A. reacting the imidazole carbonyl-coupled reactive intermediate (3) obtained in Scheme 1 with amine of formula R₁—NH₂ (III) at a temperature in the range of 45-50° C. for a period in the range of 4-5 hours to obtain amidino urea (15); wherein, R₁ is selected from the group of below compounds of formulae a, b, c and d;

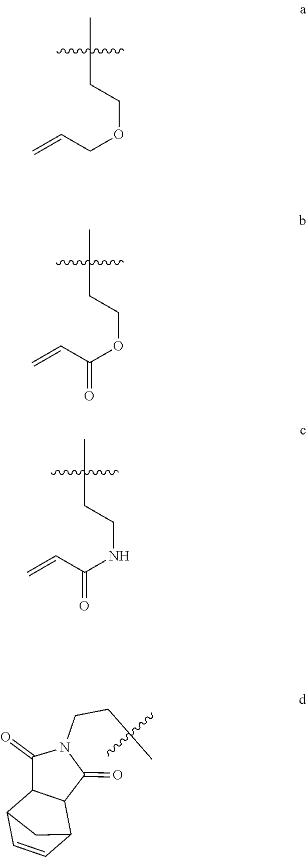

B. cyclizing the amidino urea (15) with CDI at a temperature in the range of 30-35° C. for a period in the range of 45-48 hours to obtain polymer building block (16);
C. deprotecting the compound (16) at a temperature in the range of 0-4° C. for a period in the range of 40-45 minutes to obtain free amino triazine (17).

The process is depicted below in Scheme 3:

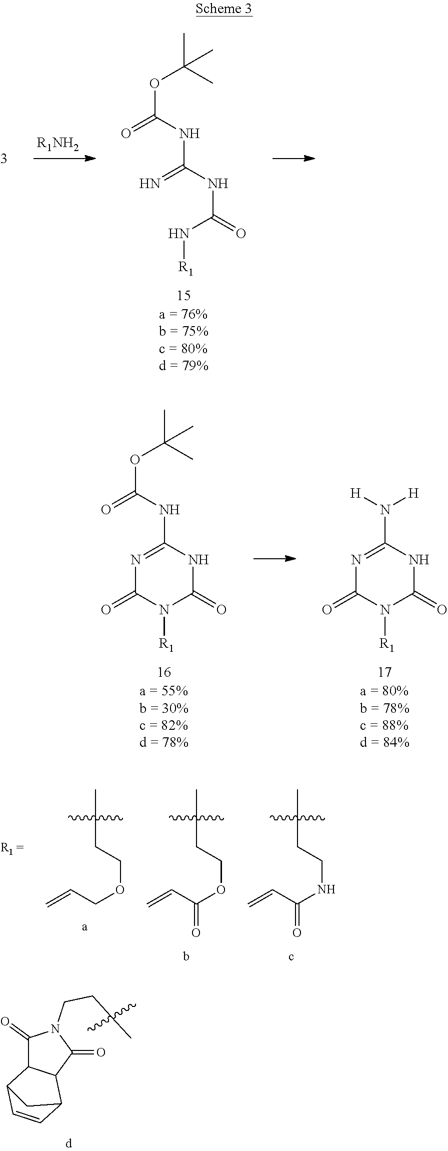

The solvent for the process is selected from polar or non-polar, protic or aprotic solvent such as lower alcohols, nitriles, ketones, halogenated hydrocarbons, TFA or combinations thereof. The base for the reaction is selected from organic base such as ethylamine, triethylamine, DIPEA, pyridine or from inorganic base such as sodium hydroxide, alkali or alkaline earth metal carbonates and bicarbonates or combination thereof.

In a preferred embodiment, the free amino triazine compound (17) comprises of:
3-(2-(allyloxy) ethyl)-6-amino-1,3,5-triazine-2,4(1H,3H)-dione (17a); 2-(4-amino-2,6-dioxo-3,6-dihydro-1,3,5-triazin-1(2H)-yl)ethyl acrylate (17b); N-(2-(4-amino-2,6-dioxo-3,6-dihydro-1,3,5-triazin-1(2H)-yl) ethyl) acrylamide (17c); and 2-(2-(4-amino-2,6-dioxo-3,6-dihydro-1,3,5-triazin-1(2H)-yl)ethyl)-3a, 4,7,7a-tetra hydro-1H-4,7-methanoisoindole-1,3(2H)-dione(17d).

In another embodiment, the building block (16) is subjected to covalent polymerization. On deprotection after copolymerization, the Janus G-C nucleobase being self-complementary triggers 3+3 type H-bonded supramolecular polymerization, which substantially influences the overall property of the polymers.

In yet another embodiment, the compound (Id) shown in Scheme 1 and polymer monomer building block (17b) of Scheme 3 are crystallized from hot aqueous methanol containing traces of HCl and hot DMSO respectively. Analysis of their single-crystal X-ray structure (FIG. 1) revealed the anticipated 3+3 repeating H-bonding pattern. Each molecule is held from both sides by two sets of DDA-AAD-type triple hydrogen-bonding arrays—reminiscent of the guanine-cytosine G≡C-type triple H-bonding pattern seen in nucleic acids. H-bonding parameters of 6d and 17b are comparable to that of native G-C base pairs. Owing to self-complementarity, the DDA-AAD-type triple hydrogen bonding leads to supramolecular polymer formation, as expected. A notable feature of this supramolecular assembly is that unlike the CA-MA motif which often leads to the formation of mixtures of cyclic rosette/crinkled tape/linear self-assembled structures, the Janus G-C nucleobase of the present invention leads to the formation of a single set of supramolecular polymers owing to the orthogonal positioning of the DDA-AAD H-bonding arrays within the molecule. The Janus G-C nucleobase of the present invention holds promise for its application in sensitive areas wherein material homogeneity is necessary.

### EXAMPLES

Following examples are given by way of illustration and therefore should not be construed to limit the scope of the invention.

Experimental

All reagents were purchased of highest commercial quality and used without further purification unless otherwise stated. All reactions were carried out with laboratory-grade solvent and purity of intermediates and final compounds were checked on pre-coated silica gel G254 TLC plates (Merck). Visualization of spot on TLC was achieved by the use of UV light (254 nm) and ninhydrin test. All synthesized compounds were purified on silica gel column chromatographic by using of 100-200, 230-400 Mesh Silica gel.

[A]: Synthesis of the Compounds of General Formula (I)

### Example 1: Synthesis of the Compound (2) N-tert-Butoxycarbonylguanidine

The synthesis of compound 2 was as per the earlier reported procedure. (Tetrahedron 2010, 66, 6224-6237). Accordingly, Guanidinium chloride monohydrate (23 g, 1 equiv., 240 mmol) was dissolved in water (48 mL), added NaOH (19.2 g, 2 equiv. 481 mmol) and stirred at 0° C. for 15 min., followed by addition of a solution of di-tert-butyl dicarbonate (13.1 g, 0.25 equiv., 60 mmol) in acetone (200 mL) and stirred at 30° C. for an additional 10 hour. The solvent was removed under vacuum and the resultant residue was dissolved in water (30 mL) and extracted with ethyl acetate (3×100 mL). The organic layer was washed with brine solution, dried (Na₂SO₄), filtered and concentrated in vacuo to dryness. The resultant residue was suspended in cold diethyl ether and solid was filtered under vacuum to afford compound 2 as a white powder.

Yield: 80%; R_{f}=0.2 (silica gel TLC, 10% methanol in DCM); ¹H NMR (400 MHz, DMSO-d⁶) δ 6.80 (bs, 4 H), 1.34 (s,9 H); ¹³C NMR (101 MHz, DMSO-d⁶) δ 162.97, 162.27, 75.07, 27.83, 27.83, 27.83; HRMS (ESI-TOF) m/z: [M+H]⁺ Calcd for C₆H₁₄N₃O₂ 160.1081; Found 160.1079; [M+Na]⁺ Calcd for C₆H₁₃N₃O₂ Na 182.0900; Found 182.0898.

### Example 2: Synthesis of Compound 3

N-Boc-guanidine 2 (14 g, 87.1 equiv., 95 mmol, 1 equiv) was suspended in dichloromethane (50 mL), added 1,1-carbonyldiimidazole (CDI) (15.69 gm, 1.1 equiv., 96.94 mmol) and solution was stirred at 30° C. for 5 hour. The resultant solution washed with water (3×30 mL), dried (Na₂SO₄) and concentrated under vacuum to afford 3 as crystalline white solid, which was carried forward for the next step.

Yield: 20 g, 90%, R_{f}=0.3 tailing (silica gel TLC, 60% ethyl acetate in pet ether); mp. 155-160° C.; ¹H NMR (400 MHZ, CDCl₃) δ 11.10 (bs., 1H), 9.29 (bs., 1H), 8.91 (bs., 1H), 8.56 (s, 1H), 7.56 (s, 1H), 7.00 (s, 1H), 1.56 (s, 9H); ¹³C NMR (101 MHz, CDCl₃) δ 160.85, 157.91, 153.77, 137.83, 128.82, 117.01, 83.60, 28.06, 28.06, 28.06; HRMS (ESI-TOF) m/z: [M+H]⁺ Calcd for C₁₀H₁₆N₅O₃ 254.1248; Found 254.1243.

### Example 3: Synthesis of Compound (4a)

The compound 3 (1 equiv., 2.00 gm, 7.9 mmol) was dissolved in acetonitrile (50 mL), added benzylamine (1.2 equiv., 1.01 g, 9.52 mmol) and stirred at 50° C.for 4 hour. The reaction mixture was cooled at 37° C. and concentrated under vacuo. The resultant residue was dissolved in ethyl acetate (100 mL) and subsequently washed with diluted KHSO₄ solution, brine solution and dried (Na₂SO₄). The organic layer was concentrated under vacuum to afford 4a as white solid.

Yield: 87%; mp. 90-100° C.; R_{f}=0.5 (silica gel TLC, 70% ethyl acetate in pet ether); ¹H NMR (400 MHz, CDCl₃) δ 8.89 (bs, 3 H) 7.77 (bs, 1 H) 7.33-7.29 (m, 5 H) 4.41-4.39 (d, J=5.34 Hz, 2 H) 1.53 (s, 9 H); ¹³C NMR (101 MHz, CDCl₃) δ 155.21, 154.22, 137.28, 128.79, 128.69, 128.57, 127.73, 127.59, 127.35, 86.09, 43.99, 27.94, 27.94, 27.94; HRMS (ESI-TOF) m/z: [M+H]⁺ Calcd for C₁₄H₂₁N₄O₃ 293.1608; Found 293.1601; [M +Na]⁺ Calcd for C₁₄H₂₀N₄O₃ Na 315.1428; Found 315.1419.

### Example 4: Synthesis of Compound (4b)

The synthetic method of 4a was adopted to synthesize 4b; white solid.

Yield: 84%; mp.100-110° C.; R_{f}=0.5 tailing (silica gel TLC, 80% ethyl acetate in pet ether); ¹H NMR (400 MHZ, CDCl₃) δ 10.12 (bs., 2 H), 7.55 (bs., 1H), 6.98 (bs., 1H), 3.05-3.03 (s, 2H), 1.53 (s, 9H), 0.94 (s, 9H); ¹³C NMR (101 MHz, CDCl₃); δ159.42, 154.81, 154.01, 86.27, 51.36, 32.09, 27.84, 27.84, 27.84, 27.15, 27.15, 27.15; HRMS (ESI-TOF) m/z: [M+H]⁺Calcd for C₁₂H₂₅N₄O₃ 273.1921; Found 273.1914; [M+Na]⁺Calcd for C₁₂H₂₄N₄O₃ Na 295.1741; Found 295.1733.

### Example 4: Synthesis of Compound (4c)

Tert-butyl (2-(benzyloxy) ethyl) carbamate (5 g, 1 equiv, 19.89 mmol, which was synthesized as per earlier reported procedure (J. Pept. Sci. 2009, 15, 366-368.) was dissolved in 50% solution of TFA in DCM (50 mL) and stirred at 0° C., for 45 min. The solvent was evaporated under vacuum and co-evaporated with toluene (2×20 mL) to take off the residual TFA from reaction mixture to afford TFA salt as a semisolid. The resultant TFA salt (1 equiv., 5.2 g, 1 equiv., 19.60 mmol) was dissolved in acetonitrile (40 mL), added compound 3 (1.1 equiv., 5.43 g, 21.56 mmol) and DIPEA (3 equiv., 10.22 mL, 58.81 mmol) and stirred at 50° C. for 4 h.

The resultant reaction mixture was cooled at 37° C. and concentrated under vacuum. The residue was dissolved in ethyl acetate (100 mL) and subsequently washed with dilute KHSO₄ solution, brine solution, dried (Na₂SO₄) and concentrated under vacuum for dryness to obtain crude 4c as white solid, which was purified by column chromatography using 10 to 90% ethyl acetate in pet ether. The resultant white solid was triturated using cold 50% diethyl ether in pet-ether (10 mL) to afford 4c as pale semisolid.

Yield: 88%; R_{f}=0.5 (silica gel TLC, 70% ethyl acetate in pet ether); ¹H NMR (400 MHZ, CDCl₃) δ 9.31 (bs., 1 H), 8.17 (bs., 1 H), 7.33-7.28 (m, 5 H), 6.38 (bs., 1 H), 4.51 (s, 2 H), 3.54-3.52 (m, 2 H), 3.40-3.38 (m, 2 H), 1.47 (s, 9 H); ¹³C NMR (101 MHz, CDCl₃) δ 158.08, 157.47, 152.96, 137.88, 128.41, 128.41, 128.41, 127.77, 127.73, 81.97, 73.15, 69.21, 39.84, 28.09. 28.09, 28.09; HRMS (ESI-TOF) m/z: [M+H]⁺ Calcd for C₁₆H₂₅N₄O₄ 337.1870; Found 337.1867.

### Example 5: Synthesis of Compound (4d)

The synthetic method of 4a was adopted to synthesize 4d. The benzyl (2-aminoethyl) carbamate was synthesized as per earlier reported procedure (J. Pept. Sci. 2009, 15, 366-368). The resulting residue was directly purified by column chromatography using 100-200 mesh size and mobile 0-10% MeOH in dichloromethane. The solvent was removed under vacuum and residue was triturated with diethyl ether to afford 4d as off-white solid.

Yield: 85%; mp. 80-90° C.; R_{f}=0.3 (silica gel, TLC, 10% methanol in DCM); ¹H NMR (400 MHz, CDCl₃) δ 8.23 (bs., 1 H), 7.30 (m, 5 H), 6.55 (m, 1 H), 6.36 (bs, 1 H), 5.80 (bs., 1 H), 5.10 (bs, 1H), 5.06 (m., 2 H), 3.27 (m., 4 H), 1.48 (m, 9 H); ¹³C NMR (101 MHz, CDCl₃) δ 162.45, 158.21, 157.13, 157.13, 136.68, 128.65, 128.65, 128.23, 128.23, 128.23, 82.40, 66.85, 41.55, 39.95, 28.27, 28.27, 28.27; HRMS (ESI-TOF) m/z: [M+H]⁺Calcd for C₁₇H₂₆N₅O₅ 380.1928; Found 380.1924; [M+Na]⁺ Calcd for C₁₇H₂₅N₅O₅Na 402.1748; Found 402.1737.

### Example 5: Synthesis of Compound (4e)

Compound 3 (2.00 gm, 1 equiv., 7.9 mmol) was dissolved in acetonitrile (30 mL) were added Gly-OBn. PTSA (1.2 equiv., 1.07 gm, 9.48 mmol, which was synthesized by previously reported procedure (J. Org. Chem. 2009, 74, 8988-8996.) and DIPEA (2.74 mL, 2 equiv. 15.81 mmol) and stirred at 50° C.for 4 hours. The reaction mixture was cool at 37° C. and diluted with ethyl acetate (70 mL) and subsequently washed with dilute KHSO⁴ solution, brine solution, dried (Na₂SO₄). The resultant organic layer was concentrated under vacuum to dryness and solid residue triturated with diethyl ether to afford 4e as white solid.

Yield: 80%; mp. 110-115° C.; R_{f}=0.4 (silica gel TLC, 80% ethyl acetate in pet ether); ¹H NMR (400 MHz, DMSO-d⁶) δ 10.87 (bs., 1 H), 9.29 (bs., 1 H), 8.69 (bs., 1 H), 7.39-7.38 (m, 5 H), 5.17 (s, 2 H), 4.00-3.99 (d, J=6.1 Hz, 2 H), 1.49 (s, 9 H); ¹³C NMR (101 MHZ, DMSO-d⁶) δ 169.08, 154.07, 153.80, 151.92, 135.53, 128.26, 128.26, 127.97, 127.81, 125.30, 83.60, 65.53, 41.17, 28.35, 28.35, 28.35; HRMS (ESI-TOF) m/z: [M+H]⁺Calcd for C₁₆H₂₃N₄O₅ 351.1663; Found 351.1653; [M+Na]⁺ Calcd for C₁₆H₂₂N₄O₅Na 373.1482; Found 373.1470.

### Example 6: Synthesis of Compound (4f)

Benzyl N⁶-((benzyloxy) carbonyl)-L-lysinate TFA salt (2.1 g, 1 equiv., 4.33 mmol, which was synthesized as per previously reported method (J. Bioorg. Med. Chem. Lett. 2020, 30, 127039.) was dissolved in acetonitrile, added compound 3 (1.20 g, 1.1 equiv., 4.76 mmol) and DIPEA (2.26 mL, 3 equiv., 13.00 mmol), and stirred at 50° C. for 4-5 h. The resultant solution was cooled at 37° C.and concentrated under vacuum. The residue was diluted with ethyl acetate (100 mL) and subsequently washed with dilute KHSO₄ solution, brine solution, dried (Na₂SO₄). The solvent was evaporated under reduced pressure to afford 4f as white solid.

Yield: 87%; mp. 80-90° C.; R_{f}=0.4 (silica gel TLC, 80% ethyl acetate in pet ether); ¹H NMR (500 MHz, DMSO-d⁶) δ 9.82 (bs., 1H), 8.23 (bs, 1H), 7.76 (bs, 1 H), 7.37-7.30 (m, 10 H), 7.25 (t, J=5.5 Hz, 1 H), 5.17-5.11 (m, 2 H), 5.01 (s, 2 H), 4.20-4.16 (d, J=5.3 Hz, 1 H), 3.36 (s, 1 H), 2.96 (q, J=6.6 Hz, 2 H), 1.74-1.65, (d, J=8.0 Hz, 2 H), 1.46-1.36 (m, 11 H), 1.31-1.29 (m, 2 H); ¹³C NMR (125 MHz, DMSO-d⁶) δ 172.37, 157.79, 156.25, 137.44, 136.13, 128.58, 128.58, 128.50, 128.50, 128.50, 128.21, 128.21, 127.89, 127.89, 127.64, 127.89, 78.76, 65.10, 64.29, 59.92, 52.82, 30.74, 29.13, 28.07, 28.07, 28.07, 22.66, 14.25; HRMS (ESI-TOF) m/z: [M+H]⁺ Calcd for C₂₈H₃₈N₅O₇ 556.2771; Found 556.2766. Note: δ 4.10, 2.07,1.27 are residual peak of ethyl acetate.

### Example 7: Synthesis of Compound (4g)

Benzyl N²-(tert-butoxycarbonyl)-N^{\dot{ω}}) ((2,2,4,6,7-pentamethyl-2,3-dihydrobenzofuran-5-yl) sulfonyl)-L-argininate (5.00 gm, 1 equiv., 8.11 mmol, which was synthesized as per previously reported method (J. Bioorg. Med. Chem. Lett. 2020, 30, 127039.) was dissolved 50% TFA in DCM (30 mL) and stirred at ice-bath temperature for 45 min. The solvent was removed under vacuum and remaining TFA was stripped off by co-evaporating with toluene (2×20 mL) to afford TFA salt as a semisolid that was carried forward for next step. The resultant TFA salt (5 gm, 1 equiv., 7.92 mmol, was dissolved in acetonitrile (40 mL), added compound 3 (2.69 g, 1.1 equiv., 8.72 mmol) and DIPEA (4.13 mL, 3 equiv., 23.78 mmol), and stirred at 50° C. for 4-5 h. The resultant solution was cooled at 37° C. and concentrated under vacuum. The residue was diluted with ethyl acetate (150 mL) and subsequently washed with dilute KHSO⁴ solution, brine solution, dried (Na₂SO₄). The solvent was evaporated under reduced pressure to afford 4g as a white solid:

Yield: 80%; mp. 90-100° C.; R_{f}=0.3 (silica gel TLC, 80% ethyl acetate in pet ether); ¹H NMR (400 MHz, DMSO-d⁶) δ 9.22 (bs., 1 H), 8.61 (bs., 1 H), 8.25-8.16 (bs., 1 H), 7.43-7.42 (m, J=5.4 Hz, 5 H), 6.86 (bs., 1 H), 6.49 (bs., 2 H), 5.14 (bs., 2 H), 4.22 (d, J=5.3 Hz, 1 H), 3.44 (bs, 1 H), 3.03-305 (dd, J=6.1, 10.2 Hz, 2 H), 2.93 (s, 2 H), 2.48 (s, 3 H), 2.42 (s, 3 H), 1.99 (s, 4 H), 1.68 (bs., 1 H), 1.47 (s, 9 H), 1.44-1.35 (m, 8 H); ¹³C NMR (101 MHZ, DMSO-d⁶) δ 172.68, 171.86, 170.81, 157.91, 156.66, 155.59, 137.72, 136.13, 134.64, 131.87, 128.93, 128.93, 128.60, 128.60, 128.37, 124.80, 116.74, 86.74, 82.92, 66.7, 60.23, 53.11, 42.92, 28.75, 28.75, 28.12, 28.12, 28.12, 21.00, 19.43, 18.08, 14.55, 12.72; HRMS (ESI-TOF) m/z: [M+H]⁺Calcd for C₃₃H₄₈N₇O₈S 702.3280; Found 702.3274; [M+Na]⁺Calcd for C₃₃H₄₇N₇O₈SNa 724.3099; Found 724.3082.

### Example 8: Synthesis of Compound (5a)

Compound 4a (2 g, 5.27 mmol, 1 equiv.), was dissolved in acetonitrile (25 mL), added NaHCO₃ (0.886 g, 10.5 mmol, 1.5 equiv) and CDI (1.28 g, 7.91 mmol, 1.2 equiv.) and stirred at 35° C. for 48 hours. The resultant precipitate was filtered under vacuum and precipitate was suspended in ethyl acetate (100 mL) and acidified with dilute KHSO₄ solution organic layer was washed with brine solution, dried (Na₂SO₄) and concentrated under vacuum to dryness. The resulting white solid was washed with diethyl ether to afford 5a as a white solid.

Yield: 70%; mp. >400° C.; R_{f}=0.5 (silica gel TLC, 10% methanol in DCM); ¹H NMR (400 MHz, CDCl₃) δ 11.24 (bs, 1 H), 10.29 (bs, 1 H), 7.48 (d, J=6.10 Hz, 2 H), 7.28 (s, 3 H), 5.05 (s, 2 H), 1.49 (s, 9 H); ¹³C NMR (101 MHz, CDCl₃) δ 154.45, 154.09, 152.82, 148.99, 136.13, 129.06, 129.06, 128.41, 128.41, 127.85, 85.50, 44.74, 27.80, 27.80, 27.80; HRMS (ESI-TOF) m/z: [M+H]⁺Calcd for C₁₅H₁₉N₄O₄ 319.1401; Found 319.1393; [M+Na]⁺Calcd for C₁₅H₁₈N₄O₄ Na 341.1220; Found 341.1212. Note: Feebly UV active but ninhydrin active with green color on long heating.

### Example 9: Synthesis of Compound (5b)

The synthetic method of 5a was adopted to synthesize 5b as white solid. Yield: 66%; mp. >400° C.; R_{f}=0.5 (silica gel TLC, 10% methanol in DCM); ¹H NMR (400 MHz, CDCl₃) δ 11.17 (bs., 1 H), 9.82 (bs., 1 H), 3.80 (s, 2 H), 1.53 (s, 9 H), 0.96 (s, 9 H); ¹³C NMR (101 MHz, CDCl₃) δ 154.37, 153.52, 152.71, 149.71, 85.38, 51.24, 33.97, 28.34, 28.34, 28.34, 27.88, 27.88, 27.88; HRMS (ESI-TOF) m/z: [M+H]⁺ Calcd for C₁₃H₂₃N₄O₄ 299.1714; Found 299.1712; [M+Na]⁺ Calcd for C₁₃H₂₂N₄O₄Na 321.1533; Found 321.1531. Note: Feebly UV active but ninhydrin active with green color on long heating.

### Example 10: Synthesis of Compound (5c)

The synthetic method of 5a was adopted to synthesize 5c; white solid.

Yield: 75%; mp.>250 ° C.; R_{f}=0.6 (silica gel TLC, 10% methanol in DCM); ¹H NMR (400 MHz, CDCl₃) δ 11.13 (bs., 1 H), 9.91 (bs., 1 H), 7.32-7.31 (m, 5 H), 4.53 (s, 2 H), 4.14 (m, 2 H), 3.72 (t, J=5.7 Hz, 2 H), 1.51 (s, 9 H); ¹³C NMR (101 MHz, CDCl₃) δ 154.16, 152.88, 152.88, 149.06, 137.99, 128.36, 128.36, 127.75, 127.75, 127.62, 85.39, 72.73, 66.43, 40.61, 27.81, 27.81, 27.81; HRMS (ESI-TOF) m/z: [M+H]⁺ C₁₇H₂₃N₄O₅ 363.1663; Found 363.1656; Calcd m/z: [M+Na]⁺ C₁₇H₂₂N₄O₅Na 385.1482; Found 385.1475. Note: Feebly UV active but ninhydrin active with green color on long heating.

### Example 11: Synthesis of Compound (5d)

The synthetic method of 5a was adopted to synthesize 5d; white solid.

Yield: 76%, mp.>350° C.; R_{f}=0.4 (silica gel TLC, 80% ethyl acetate in pet ether); ¹H NMR (400 MHz, CDCl₃) δ 11.05 (bs., 1H), 9.16 (bs., 1 H), 7.35-7.34 (m, 5 H), 5.33 (bs, 1 H), 5.08 (s, 2 H), 4.09-4.06 (m, 2 H), 3.54-3.50 (m, 2 H), 1.54 (s, 9 H); ¹³C NMR (101 MHz, CDCl₃) δ 156.58, 155.16, 153.69, 152.55, 149.10, 136.54, 128.45, 128.45, 128.05, 128.05, 128.05, 85.89, 66.67, 41.09, 39.80, 27.85, 27.85, 27.85; HRMS (ESI-TOF) m/z: [M+H]⁺Calcd for C₁₈H₂₄N₅O₆ 406.1721; Found 406.1716Note: ¹H NMR δ 1.53 residual CH₂Cl₂, δ 1.56 residual water. Note: Feebly UV active but ninhydrin active with green color on long heating.

### Example 12: Synthesis of Compound (5e)

The synthetic method of 5a was adopted to synthesize 5e; white solid.

Yield: 60%; mp. 280-290° C.; R_{f}=0.4 (silica gel TLC, 70% ethyl acetate in pet ether); ¹H NMR (400 MHZ, CDCl₃) δ 11.09 (bs., 2 H), 7.36-735 (d, J=3.0 Hz, 5 H), 5.19 (s, 2 H), 4.68 (s, 2 H), 1.51 (s, 9 H); ¹³C NMR (101 MHz, CDCl₃) δ 167.22, 158.72, 154.48, 152.87, 135.11, 128.64, 128.64, 128.47, 128.47, 128.26, 85.87, 67.40, 60.41, 42.24, 27.81, 27.81, 28.81; HRMS (ESI-TOF) m/z: [M+H]⁺ Calcd for C₁₇H₂₁N₄O₆ 377.1456; Found 377.1452; [M+Na]⁺ Calcd for C₁₇H₂₀N₄O₆Na 399.1275; Found 399.1271. Note: residual peak of ethyl acetate at δ 1.27, 2.07, 4.26 Note: Feebly UV active but ninhydrin active with green color on long heating.

### Example 13: Synthesis of Compound (5f)

The synthetic method of 5a was adopted to synthesize 5f; white solid.

Yield: 45%; mp. 80-90° C.; R_{f}=0.5 (silica gel TLC, 45% ethyl acetate in pet ether); ¹H NMR (400 MHz, DMSO-d⁶) δ 11.22 (bs., 1 H), 7.37-7.30 (m, 10 H), 5.23 (t, J=5.5 Hz, 1 H), 5.21-5.10 (m, 3 H), 5.07-4.99 (m, 2 H), 3.00-2.92 (m, 2 H), 2.08 (dd, J=5.9, 8.9 Hz, 1 H), 2.03-1.94 (m, 1 H), 1.47 (s, 9 H), 1.44-1.33 (m, 3 H), 1.26 (d, J=6.9 Hz, 2 H); ¹³C NMR (101 MHz, DMSO-d⁶) δ 172.51, 172.34, 169.48, 156.56, 154.56, 153.72, 137.73, 136.34, 128.85, 128.85, 128.79, 128.79, 128.56, 128.56, 128.43, 128.31, 128.20, 128.03, 83.99, 66.67, 65.57, 54.01, 29.53, 28.98, 28.05, 28.05, 28.05, 23.16, 14.87; HRMS (ESI-TOF) m/z: [M+H]⁺ Calcd for C₂₉H₃₆N₅O₈ 582.2558; Found 582.2547; Note: Feebly UV active but ninhydrin active with green color on long heating.

### Example 14: Synthesis of Compound (5g)

The synthetic method of 5a was adopted to synthesize 5g; white solid.

Yield: 49%; mp. 220-230° C.; R_{f}=0.4 (silica gel TLC, 10% methanol in DCM); ¹H NMR (400 MHz, CDCl₃) δ 11.23 (bs, 1 H), 7.89 (bs, 1 H), 7.29-7.28 (m, 5 H), 6.29-6.20 (bs., 3 H), 5.30-5.16 (m, 1 H), 5.13-5.10 (m, 3 H), 5.07 (d, J=2.3 Hz, 1 H), 3.58-3.50 (m, 2 H), 2.92 (s, 3 H), 2.54 (s, 3 H), 2.47 (s, 3 H), 2.17 (bs., 1 H), 2.06 (s, 3 H), 1.46-1.45 (d, J=5.3 Hz, 15 H); ¹³C NMR (101 MHz, CDCl₃) δ 169.01, 158.59, 156.19, 153.19, 153.16, 148.63, 138.26, 135.25, 132.18, 132.18, 128.52, 128.31, 128.31, 128.31, 128.13, 124.52, 117.37, 86.33, 67.34, 65.86, 54.22, 43.18, 40.62, 28.58, 28.58, 27.76, 27.76, 27.76, 25.69, 25.47, 19.27, 17.92, 15.27, 12.47; HRMS (ESI-TOF) m/z: [M+H]⁺ Calcd for C₃₄H₄₆N₇O₉S 728.3072; Found 728.3071; Note: Feebly UV active but ninhydrin active with green color on long heating.

### Example 15: Synthesis of 6-amino-3-benzyl-1,3,5-triazine-2,4 (1H,3H)-dione (Ia)

A mixture of 5a (101 mg, 1 equiv. 0.331 mmol) in 50% TFA in DCM (5 mL) was stirred at 0-4° C. for 45 min. The reaction mixture was concentrated in vacuo and co-evaporated with dichloromethane (2×10 mL). The resulting residue was diluted with the diethyl ether (5 mL) and solid was filtered under vacuum to afford Ia as a white solid.

Yield: 90%; mp.>400° C.; ¹H NMR (400 MHZ, DMSO-d⁶) δ 12.52 (bs, 2H), 9.22 (bs, 1H), 7.32-7.22 (m, 5 H), 4.85 (s, 2 H); ¹³C NMR (101 MHz, DMSO-d⁶) δ 159.67, 153.24, 149.05, 136.66, 129.08, 128.51, 128.51, 128.25, 128.25, 44.84; HRMS (ESI-TOF) m/z: [M+H]⁺ Calcd for C₁₀H₁₁N₄O₂⁺ 219.0877; Found 219.0876; Note: poor solubility even in hot DMSO-d⁶.

### Example 16: Synthesis of 6-amino-3-neopentyl-1,3,5-triazine-2,4 (1H,3H)-dione (Ib)

The synthetic method of Ia was adopted to synthesize Ib as a white solid.

Yield: 92%; mp.>400° C.; The product was poorly soluble in any solvent; HRMS ESI-TOF: Calcd m/z: [M+H]⁺ calculated for C₈H₁₅N₄O₂⁺ 199.1190; Found; 199.1189; M+Na]⁺ Calcd for C₈H₁₄N₄O₂ 221.1009; Found 221.1007 Note: no solubility in DMSO-d⁶.

### Example 17: Synthesis of 6-amino-3-(2-hydroxyethyl)-1,3,5-triazine-2,4 (1H,3H)-dione (Ic)

The compound 5c (1 g, 1 equiv. 2.47 mmol) was dissolved in methanol (5 mL) followed by addition of Pd/C (20 mol %) and stirred at 35° C. for 4 h under hydrogen (H₂) atmosphere. The reaction mixture was passed through a celite pad (thin pad) and pad was washed repetitively by MeOH (4×20 mL). The resultant filtrate was concentrated under vacuum to afford benzyl free intermediate (0.6 g) as a white solid. Further, the resultant solid (0.1 g, 1 equiv.) was dissolved in 50% solution of TFA in DCM and stirred at 0-4° C. for 45 min. The solvent was removed under vacuum and co-evaporated with DCM (2×5 mL) and the resultant residue was diluted with diethyl ether (10 mL) and resultant solid was washed by filtration under vacuum to afford Id as a white solid; overall yield: 80%; mp.>250° C.; ¹H NMR (400 MHZ, DMSO-d⁶) δ 7.53 (bs. 1 H), 5.01 (bs., 3 H), 3.75-3.72 (m, 2 H), 3.49-3.45 (t, J=6.5 Hz, 2 H), 3.17 (s, 1H); ¹³C NMR (400 MHz, DMSO-d⁶) δ 159.51, 155.20, 151.98, 58.14, 42.96; HRMS ESI-TOF: Calcd m/z: [M+H]⁺ Calcd for CsHON403 173.0669; Found 173.0669. Note: poor solubility in hot DMSO-d⁶.

### Example 18: Synthesis of 6-amino-3-(2-aminoethyl)-1,3,5-triazine-2,4(1H,3H)-dione (Id)

The synthetic method of Ia was adopted to synthesize Ic white solid; over all Yield: 70%; mp.>250° C.; ¹H NMR (400 MHZ, DMSO-d⁶) δ 11.54 (bs, 1H), 7.80 (bs. 2 H), 7.46 (bs., 2H), 3.93 (d, 2 H), 2.99 (d, J=6.5 Hz, 2 H); HRMS (ESI-TOF): Calcd m/z: [M+H]⁺ Calcd for C₅H₁₀N₅O₂⁺ 172.0829; Found 172.0829. [M+H]⁺ Note: poor solubility in DMSO-d⁶, δ 3.39 (bs, residual water).

### Example 19: Synthesis of Benzyl 2-(4-amino-2,6-dioxo-3,6-dihydro-1,3,5-triazin-1(2H)-yl) acetate (Ie)

The synthetic method of Ia was adopted to synthesize Ie.

Yield: 80%; mp. >250° C.; ¹H NMR (400 MHz, DMSO-d⁶) δ 7.39-7.34 (m, 5 H), 7.23 (bs., 2 H), 5.16 (s, 2 H), 4.48 (s, 2 H), ¹³C NMR (100 MHz, DMSO-d⁶) δ 168.76, 156.82, 156.82, 153.17, 136.23, 129.98, 128.67, 128.39, 128.39, 128.39, 66.72, 42.02; HRMS ESI-TOF: Calcd m/z: [M+H]⁺ C₁₂H₁₃N₄O₄⁺ 277.0931; Found 277.0931; [M+Na]⁺ Calcd for C₁₂H₁₂N₄O₄Na; 299.0756; Found 299.0751.

### Example 20: Synthesis of Benzyl 2-(4-amino-2,6-dioxo-3,6-dihydro-1,3,5-triazin-1(2H)-yl)-6-(((benzyloxy) carbonyl) amino) hexanoate (If)

The synthetic method of Ia was adopted to synthesize If as a white solid.

Yield: 78%; mp.270-280° C.; ¹H NMR (400 MHz, DMSO-d⁶) δ 7.37-7.29 (m, 10 H), 7.22 (bs., 2H), 5.19-5.15 (dd, J=5.0, 9.5 Hz, 1 H), 5.13-5.05 (m, 2 H), 4.99 (s, 2 H), 2.97 (q, J=6.6 Hz, 2 H), 2.06-1.96 (m, 2 H), 1.49-1.31 (m, 2 H), 1.20 (dd, J=6.5, 14.9 Hz, 2 H); ¹³C NMR (101 MHz, DMSO-d⁶) δ 170.14, 159.62, 156.75, 156.59, 153.30, 137.81, 136.55, 128.88, 128.88, 128.45, 128.45, 128.25, 128.25, 128.01, 128.01, 128.01, 28.01, 66.48, 65.61, 53.41, 29.60, 28.48, 23.38, 18.38; HRMS (ESI-TOF): Calcd m/z: [M+H]⁺ C₂₄H₂₈N₅O₆⁺ 482.2034; Found 482.2034 [M+H]⁺.

### Example 21: Synthesis of Benzyl (S)-2-(4-amino-2,6-dioxo-3,6-dihydro-1,3,5-triazin-1(2H)-yl)-5-guanidino pentanoate (Ig)

Deprotection of 5g using 95% TFA-DCM afforded Ig as an insoluble material. Owing to poor solubility, it could not be satisfactorily characterized. Yield: 62%; mp. 175-185° C.; HRMS ESI-TOF: Calcd m/z: [M +H]⁺ C₁₆H₂₂N₇O₄⁺ 376.1728; Found 376.1724.

[B] Synthesis of Triazine-Based Janus G-C Nucleobase Containing PNA Building Blocks

### Example 22: Synthesis of Compound (12a)

Compound 5e was dissolved in methanol (5 mL) added Pd/C (20 mol %) and stirred at 35° C. under H₂ atmosphere for 5 h. The resultant reaction mixture was passed through thin celite pad and the filtrate was concentrated under vacuo to afford 12a as a white solid.

Yield: 90%; mp. >250° C.; ¹H NMR (400 MHz, DMSO-d⁶) δ 11.79 (m, 3 H), 4.37 (s, 2 H), 1.48 (s, 9 H); ¹³C NMR (101 MHz, DMSO-d⁶) δ168.82, 168.82, 153.76, 153.76, 153.12, 83.29, 41.65, 27.44, 27.44, 27.44; HRMS (ESI-TOF): Calcd m/z: [M+H]⁺ C₁₀H₁₅N₄O₆ 287.0986; Found 287.0981. Note: Residual ethyl acetate peak at δ 1.07.

### Example 23: Synthesis of Compound (12b)

The synthetic method of 12a was adopted to synthesize 12b.

Yield: 86%; mp.220-230° C.; ¹H NMR (400 MHz, DMSO-d⁶) δ 11.35 (bs., 2 H), 6.59 (bs., 1 H), 6.38 (bs., 1 H), 5.02 (bs., 1 H), 3.37 (m, 3 H), 3.02 (d, J=5.3 Hz, 2 H), 2.96 (s, 2 H), 2.47 (s, 3 H), 2.42 (s, 3 H), 2.03-1.98 (m, 4 H), 1.48 (s, 9H), 1.41 (s, 6 H), 1.35 (d, J=10.7 Hz, 2 H); ¹³C NMR (101 MHz, DMSO-d⁶) δ 166.04, 161.29, 157.44, 156.06, 137.27, 134.11, 131.42, 124.34, 116.27, 112.15, 110.0, 86.31, 64.94, 61.18, 59.77, 42.47, 28.33, 28.33, 27.64, 27.64, 27.64, 25.31, 18.95, 17.58, 15.19, 14.10, 12.28; HRMS (ESI-TOF): Calcd m/z: [M+H]⁺Calcd for C₂₇H₄₀N₇O₉S 638.2603; Found 638.2604 [M+H]⁺.

### Example 24: Synthesis of Compound (13a)

Compound 12a (0.3 g, 1.05 mmol, 1 equiv.) was dissolved in dry DMF (5 mL) and added HBTU (0.597 g, 1.57 mmol, 1.5 equiv), HOBt (212 mg, 1.57 mmol, 1.5 equiv), DIPEA (0.364 mL, 2.09 mmol, 2 equiv) and stirred at 0° C. After stirring for 10 min. at 0° C., compound (11) (541 mg, 1.2 equiv., 1.26 mmol, which was synthesized as per earlier reported procedure⁴ was added and the reaction mixture was stirred at 35° C. for an additional 12 hour. The resultant solution was diluted with ice cold water and the solid precipitate was filtered under vacuum. The solid residue was dissolved in ethyl acetate and subsequently washed with KHSO₄ solution, NaHCO₃ solution and brine solution, dried (Na₂SO₄) and concentrated under vacuo to get white solid product which was purified by chromatography on silica and mobile phase was 50-100% EtOAc in pet-ether to afford 13a as a white solid:

Yield: 95%; mp. 180-190° C.; ¹H NMR (400 MHz, DMSO-d⁶) δ 11.41 (bs, 1H), 7.89-7.87 (d, J=7.4 Hz, 2 H), 7.69-7.67 (d, J=7.4 Hz, 2 H), 7.40-7.32 (m, 10 H), 5.12 (s, 1 H), 4.69 (s, 1 H), 4.55 (s, 1 H), 4.43 (bs., 1 H), 4.34-4.32 (d, J=6.9 Hz, 1 H), 4.29-4.23 (dd, J=6.8, 18.8 Hz, 2 H), 4.13 (s, 1 H), 3.50-4.47 (t, J=6.3 Hz, 2 H), 3.35-3.33 (m, 2 H), 3.25-3.24 (d, J=5.9 Hz, 2 H), 1.48 (s, 9 H); ¹³C NMR (100 MHz, DMSO-d⁶) δ 169.29, 168.87, 166.77, 166.62, 156.28, 153.72, 143.81, 140.65, 135.68, 128.35, 128.35, 128.09, 128.09, 127.95, 127.95, 127.78, 127.78, 127.49, 126.97, 125.07, 120.01, 83.33, 66.44, 65.85, 65.43, 59.66, 46.80, 46.78, 46.64, 40.07, 27.54, 27.54, 27.54, 20.96, 20.67, 14.00; HRMS (ESI-TOF): Calcd m/z: [M+H]⁺ Calcd for C₃₆H₃₉N₆O₉ 699.2779; Found 699.2776; [M+Na]⁺ Calcd for C₃₆H₃₈N₆O₉Na 721.2598; Found 721.2592.

### Example 25: Synthesis of Compound (13b)

The synthetic method of 13a was adopted to synthesize 13b as a white solid:

Yield: 78%; mp. 210-220° C.; ¹H NMR (400 MHz, DMSO-d⁶) δ 11.24 (bs., 1 H), 7.86 (d, J=7.1 Hz, 2 H), 7.67 (d, J=6.6 Hz, 2 H), 7.38-7.30 (m, 10 H), 7.16 (bs., 1 H), 6.68 (bs., 1 H), 6.39 (bs., 1 H), 5.20-5.14 (m, 1 H), 5.87-4.84 (m, 2 H), 4.28 (d, J=6.8 Hz, 2 H), 4.22-4.19 (m, 2 H), 4.11 (d, J=5.8 Hz, 1 H), 4.05-3.89 (m, 2 H), 3.34 (bs., 4 H), 3.20-3.19 (m, 1 H), 3.14 (bs., 2 H), 3.08-2.98 (m, 2 H), 2.94 (s, 2 H), 2.69 (s, 1 H), 2.47 (s, 2 H), 2.41 (s, 3 H), 2.01-1.97 (m, 3 H), 1.44 (s, 6 H), 1.41-1.37 (m, 9 H); ¹³C NMR (101 MHz, DMSO-d⁶) δ 169.98, 157.89, 156.54, 144.35, 141.18, 137.74, 135.61, 131.90, 128.88, 128.88, 128.73, 128.73, 128.67, 128.52, 128.52, 128.32, 128.32, 128.05, 128.05, 127.52, 127.52, 125.63, 124.80, 120.55, 116.73, 86.74, 67.06, 66.31, 65.83, 60.23, 52.49, 48.94, 47.18, 42.91, 38.16, 30.57, 29.46, 28.76, 28.05, 28.05, 28.05, 28.00, 28.00, 26.42, 21.55, 21.24, 19.42, 18.06, 17.69, 14.79, 14.55, 14.44, 12.73; HRMS (ESI-TOF): Calcd m/z: [M+H]⁺Calcd for C₅₃H₆₄N₉O₁₂S 1050.4390; Found 1050.4382.

### Example 26: Synthesis of Compound (14a)

Compound 12a (0.3 g, 1.05 mmol, 1 equiv.) was dissolved in DMF (5 mL) and added HBTU (0.597 g, 1.57 mmol, 1.5 equiv), HOBt (212 mg, 1.57 mmol, 1.5 equiv), DIPEA (0.364 mL, 2.09 mmol, 2 equiv) and stirred for 5 min, at 0° C. Ethyl (2-(((benzyloxy) carbonyl) amino) ethyl) glycinate (8) (Cbz-AEG-OEt) (0.352 g, 1.26 mmol, 1.2 equiv, synthesized as per earlier procedure 4 was added and the reaction mixture was stirred at 35° C. for an additional 16 hour. The resultant solution was diluted with ice-cold water and solid precipitate was filtered under vacuum. The solid residue was dissolved in ethyl acetate and subsequently washed with dilute KHSO⁴ solution, NaHCO₃ solution and brine solution, dried (Na₂SO₄) and concentrated under vacuo to get white solid crude product which was purified by column chromatography silica gel (230-400) and the mobile phase was EtOAc (50-90%) in pet-ether to afford 14a as a white solid:

Yield: 79%; mp, 150-160° C.; ¹H NMR (400 MHz, CDCl₃) δ 11.07 (bs, 1H), 7.39-7.30 (m, 5 H), 6.00 (bs., 1 H), 5.13 (m, 2 H), 5.09 (m, 2H), 4.71 (m., 1 H), 4.58 (s, 1 H), 4.25-4.23 (m, 2 H), 4.00 (bs., 1 H), 3.55 (bs., 2 H), 3.48-3.34 (m, 2 H), 1.51 (s, 9 H), 1.29-1.23 (m, 3 H); ¹³C NMR (101 MHz, CDCl₃) δ 169.61, 168.86, 166.78, 156.85, 153.97, 152.71, 136.62, 128.43, 128.21, 127.99, 127.99, 85.78, 66.87, 66.62, 62.20, 61.57, 49.13, 48.74, 42.00, 39.29, 27.85, 27.85, 27.85, 14.08; HRMS (ESI-TOF): Calcd m/z: [M+H]⁺ Calcd for C₂₄H₃₃N₆O₉ 549.2304; Found 549.2294.

### Example 27: Synthesis of Compound (14b)

The synthetic method of 14a was adopted to synthesize 14b as a white solid.

Yield: 72%; mp 150-160° C.. ¹H NMR (400 MHZ, DMSO-d⁶) δ 11.47 (bs, 1H), 11.04 (bs, 1H), 7.37-7.32 (m, 5 H), 7.19-7.01 (m, 1 H), 6.67 (bs, 1H), 6.37 (bs., 1 H), 5.17-5.14 (m, 1 H), 5.00-4.98 (m, 2 H), 4.08-4.08 (m, 4 H), 3.43-3.40 (dd, J=5.7, 12.7 Hz, 2 H), 3.15-3.08 (m, 3 H), 3.02-2.96 (m, 5 H), 2.47 (s, 3 H), 2.41 (s, 3 H), 2.04-1.96 (m, 4 H), 1.53-1.43 (m, 10 H), 1.41 (s, 7 H), 1.20-1.10 (m, 3 H); ¹³C NMR (101 MHz, DMSO-d⁶) δ 169.93, 168.89, 157.90, 156.57, 156.50, 137.73, 137.67, 134.65, 131.90, 128.80, 128.80, 128.21, 128.14, 128.14, 124.80, 116.72, 86.76, 84.07, 65.84, 65.68, 65.41, 61.43, 60.90, 60.23, 52.51, 47.78, 42.93, 38.83, 32.39, 31.46, 28.77, 28.77, 28.03, 28.03, 28.03, 25.94, 19.04, 18.0, 14.47, 14.21, 12.7; HRMS (ESI-TOF): Calcd m/z: [M+H]⁺Calcd for C₄₁H₅₈N₉O₁₂ 900.3920; Found 900.3925.

[C] Synthesis of Polymer Building Blocks

### Example 28: Synthesis of Compound (15a)

Tert-butyl (2-(allyloxy) ethyl) carbamate (5 g, 1 equiv. 24.87 mmol, synthesized as per earlier reported procedure (J. Pept. Sci. 2009, 15, 366-368.) was dissolved in 50% TFA in DCM and allowed to stir at 0° C. for 45 min. The resultant solution was stripped off and co-evaporated using toluene (2×20 mL) to obtain the TFA salt. The resultant TFA salt (5 g, 1 equiv. 23.25 mmol) was dissolved in acetonitrile (50 mL), added compound 3 (6.44 g, 1.1 equiv. 25.58 mmol) followed by the addition of DIPEA (8 mL, 2 equiv. 46.511 mmol) and stirred at 50° C. for 5 h. The resulting reaction mixture was cooled at 37° C. and concentrated under vacuum and the acquired residue was dissolved in ethyl acetate (150 mL) and subsequently washed with KHSO⁴ solution, brine solution, dried (Na₂SO₄). The organic layer was concentrated under vacuum to afford 15a as pale semisolid.

Yield: 76%; R_{f}=0.4 (silica gel TLC, 60% ethyl acetate in pet-ether); ¹H NMR (400 MHZ, CDCl₃) δ 9.32 (bs., 1 H), 8.09 (m, 1 H), 6.39 (m, 1 H), 5.89-5.82 (m, 1 H), 5.25-5.14 (m, 2 H), 3.97-3.96 (d, J=5.3 Hz, 2 H), 3.51-3.48 (m, 3 H), 3.37-3.35 (d, J=5.3 Hz, 2 H), 1.45 (s, 9 H); ¹³C NMR (101 MHz, CDCl₃) δ 158.06, 134.46, 117.39, 82.03, 73.37, 72.11, 70.57, 69.25, 39.85, 28.12, 28.12, 28.12; HRMS (ESI-TOF): Calcd m/z: [M+H]⁺Calcd for C₁₂H₂₃N₄O₄ 287.1714; Found 287.1710; [M+Na]⁺Calcd for C₁₂H₂₂N₄O₄Na; 309.1533 Found 309.1526.

### Example 29: Synthesis of Compound (15b)

2-((tert-butoxycarbonyl) amino) ethyl acrylate (10 g, 1 equiv. 46.46 mmol, synthesized as per earlier reported procedure (Tetrahedron, 2013, 69, 6810-6820) was dissolved in 50% TFA in DCM (40 mL) and stirred at 0° C. for 60 min, monitored by TLC. The resultant solution was stripped off and co-evaporated using toluene (2×20 mL) at 30° C. (to avoid undesired polymerization) to obtain TFA salt which was carried forward for the next step without purification. The resultant TFA salt (10 g, 1 equiv. 43.66 mmol), was dissolved in acetonitrile (80 mL) and added compound 3 (12.15 g, 1.1 equiv. 4.80 mmol) followed by addition of DIPEA (14.96 mL, 2 equiv. 8.73 mmol) and stirred at 50° C.for 5 h. The solution was cooled at 37° C. and concentrated under vacuum. The resultant residue was diluted with ethyl acetate (200 mL) and subsequently washed with dilute KHSO⁴ solution, brine solution, dried (Na₂SO₄). The solvent was removed under vacuum to afford 15b as a pale semi-solid.

Yield: 75%; R_{f}=0.5 (silica gel TLC, 60% ethyl acetate in pet-ether); ¹H NMR (500 MHZ, CDCl₃) δ 9.12, (bs, 1H), 8.32 (bs., 1 H), 6.32 (d, J=17.5 Hz, 1 H), 6.09 (bs, 1H), 6.06 (m, 1 H), 5.80-5.78 (d, J=10.7 Hz, 1 H), 4.19 (bs., 2 H), 3.45 (bs., 2 H), 1.99 (bs, 1H), 1.42 (d, J=1.5 Hz, 9 H); ¹³C NMR (101 MHz,CDCl₃) δ 166.02, 158.34, 131.22, 128.02, 81.95, 63.46, 60.34, 49.41, 38.84, 28.08, 28.08, 28.08; HRMS (ESI-TOF): Calcd m/z: [M+H]⁺ Calcd for C₁₂H₂₁N₄O₅ 301.1506; Found 301.1510; [M+Na]⁺ Calcd for: C₁₂H₂₀N₄O₅Na 323.1326; Found 323.1330.

### Example 30: Synthesis of Compound (15c)

Tert-butyl (2-acrylamidoethyl) carbamate (4.28 gm, 1 equiv., 20.00 mmol which was synthesized as per earlier reported procedure (ACS Macro Lett. 2018, 75, 592-597.) was deprotected by using 50% solution of TFA in DCM (40 mL) at ice temperature for 45 min. The resultant solution was stripped off and co-evaporated at 30° C.(to avoid undesired polymerization) using toluene (2×20 mL) to obtain the TFA salt (4.52 gm, 1 equiv., 19.84 mmol) as a semisolid, which was dissolved in acetonitrile, added compound 3 (5 gm, 1 equiv., 19.84 mmol) and DIPEA (3 equiv., 10.34 mL, 59.52, mmol), and the solution was stirred at 50° C. for 5 hours. The resultant solution was cooled at 37° C., filtered through cotton pad and filtrate was diluted with ethyl acetate (100 mL) and subsequently washed with dilute KHSO₄ solution, brine solution, dried (Na₂SO₄). The solvent was removed under vacuum to afford 15c as a white solid.

Yield: 80%; mp. 95-105° C.; R_{f}=0.3 (silica gel TLC, 80% ethyl acetate in pet-ether); ¹H NMR (400 MHZ, CDCl₃), δ 8.23 (bs, 3H), 7.06 (bs., 2H), 6.59 (bs, 3H), 6.27-6.09 (m, 2H), 5.61 (d, J=10.7 Hz, 1H), 3.46-3.38 (m, 4H), 1.48 (s, 9 H); ¹³C NMR (101 MHz, CDCl₃) δ 171.24, 166.44, 157.74, 130.92, 126.30, 82.21, 60.43, 40.63, 39.44, 28.10, 28.10, 28.10; HRMS (ESI-TOF): Calcd m/z: [M+H]⁺Calcd for C₁₂H₂₂N₅O₄ 300.1666; Found 300.1662.[Note δ 1.26, δ 2.05, δ 4.12 are residual peak of ethyl acetate]

### Example 31: Synthesis of Compound (15d)

The synthetic method of 4a was adopted to synthesize 15d. The mono-endo-amine was synthesized via earlier reported procedure. (J. Org. Chem. 2004, 69, 8340-8344.)

Yield: 79%; R_{f}=0.4 (silica gel TLC, 60% ethyl acetate in pet-ether); ¹H NMR (400 MHZ, CDCl₃) δ 9.29 (m, 1 H), 8.31 (m, 1 H), 6.08 (s, 2 H), 5.89 (bs, 1 H), 3.51-3.48 (m, 2 H), 3.34 (bs., 2 H), 3.27 (q, J=6.1 Hz, 2 H), 3.23 (bs., 2 H), 1.71-1.68 (m, 1 H), 1.51 (d, J=9.2 Hz, 1 H), 1.45 (s, 9 H), ¹³C NMR (101 MHz, CDCl₃) δ 177.93, 177.93, 161.88, 158.24, 157.79, 134.53, 134.53, 82.42, 60.45, 53.53, 52.25, 45.87, 44.92, 37.98, 37.98, 28.19, 28.19, 28.19; HRMS (ESI-TOF): Calcd m/z: [M+H]⁺ Calcd for C₁₈H₂₆N₅O₅ 392.1928; Found 392.1919.

### Example 32: Synthesis of Compound (16a)

The synthetic method of 5a was adopted to synthesize 16a.

Yield: 65%; mp. 130-135° C.; R_{f}=0.45 (silica gel TLC, 60% ethyl acetate in pet-ether); 1H NMR (400 MHz, CDCl₃) δ 9.98 (bs., 1 H), 5.86 (tdd, J=5.6, 11.0, 16.7 Hz, 1 H), 5.26-5.13 (m, 2 H), 4.10 (t, J=5.7 Hz, 2 H), 3.98 (d, J=6.1 Hz, 2 H), 3.65 (t, J=5.7 Hz, 2 H), 1.51 (s, 9 H); ¹³C NMR (101 MHz, CDCl₃) δ 154.29, 152.95, 151.62, 149.38, 134.54, 117.27, 85.51, 71.74, 66.28, 40.70, 27.89, 27.89, 27.89; HRMS (ESI-TOF): Calcd m/z: [M+H]⁺ Calcd for C₁₃H₂₁N₄O₅ 313.1506; Found 313.1496; [M+Na]⁺Calcd for C₁₃H₂₀N₄O₅Na 335.1326; Found 335.1315.

### Example 33: Synthesis of Compound (16b)

A mixture of 15b (2.5 g, 8.33 mmol, 1 equiv), NaHCO₃ (1.0 g, 12.5 mmol, 1.5 equiv) and CDI (1.62 g, 10 mmol, 1.2 equiv.) in 30 ml acetonitrile was stirred at 35° C. for 48 h. The resultant reaction mixture was filtered under vacuum and solid residue was suspended in ethyl acetate (100 mL) and acidified with dilute KHSO₄ solution, organic layer washed with brine solution, dried (Na₂SO₄), filtered and concentrated under vacuum at 30° C. (to avoid undesired polymerization) and resulting semisolid was purified by column chromatography using ethyl acetate 0-80% in petroleum ether. The solvent was removed under vacuum at 30° C. to get viscous oil which was kept in refrigerator overnight to afford 16b as a white solid. Yield: 30%; mp. 95-105° C.; R_{f}=0.6 (silica gel TLC, 60% ethyl acetate in pet-ether); ¹H NMR (400 MHz, CDCl₃) δ 11.16 (bs, 1 H), 9.85 (bs, 1 H), 6.41-6.37 (m, 1 H), 6.12-6.09 (m, 1 H), 5.85-5.82 (m, 1 H), 4.40-4.39 (m, 2 H), 4.23-4.20 (m, 2 H), 1.52 (s, 9 H) ¹³C NMR (101 MHz, CDCl₃) δ 166.23, 154.67, 154.40, 153.01, 149.19, 131.63, 128.26, 86.02, 61.50, 40.74, 28.11, 28.11, 28.11; HRMS (ESI-TOF): Calcd m/z: [M+H]⁺Calcd for C₁₃H₁₉N₄O₆ 327.1299; Found 327.1295; [M+Na]⁺Calcd for C₁₃H₁₈N₄O₆Na 349.1119; Found 349.1115.

### Example 34: Synthesis of Compound (16c)

The synthetic method of 16b was adopted to synthesize 16c.

Yield: 82%; mp. >250° C.; R_{f}=0.3 (silica gel TLC, 10% Methanol in DCM); ¹H NMR (400 MHz, DMSO-d⁶) δ 8.17 (s, 1 H), 6.10-6.05 (m, 2 H), 5.58-5.55 (dd, J=3.1, 9.9 Hz, 1 H), 3.82-3.79 (bs, 3H), 3.35 (d, J=6.1 Hz, 2 H), 1.48 (s, 9 H); ¹³C NMR (101 MHz, DMSO-d⁶) δ 165.27, 165.27, 160.00, 159.0, 153.92, 132.10, 125.46, 83.68, 40.49, 36.40, 28.01,28.01, 28.01; HRMS (ESI-TOF): Calcd m/z:[M+H]⁺ Calcd for C₁₃H₂₀N₅O₅ 326.1459; Found 326.1452; [M+Na]⁺ Calcd for C₁₃H₁₉N₅O₅Na 348.1284; Found 348.1271.

### Example 35: Synthesis of Compound (16d)

The synthetic method of 16b was adopted to synthesize 16d.

Yield: 78%; mp.>250° C.; R_{f}=0.45 (silica gel TLC, 10% MeOH in DCM); ¹H NMR (400 MHz, CDCl₃) δ 11.04 (bs., 1 H), 9.27 (bs., 1 H), 6.07-6.06 (m, 2 H), 4.02-4.03 (m, 2 H), 3.69 (td, J=2.3, 4.6 Hz, 2 H), 3.34-3.27 (d, J=19.1 Hz, 4 H), 1.72-1.70 (m, 2H), 1.54 (m, 9 H); ¹³C NMR (100 MHz, CDCl₃) δ 177.74, 177.74, 154.50, 153.35, 152.19, 148.71, 134.08, 134.08, 85.42, 51.98, 45.60, 45.60, 45.60, 44.27, 39.87, 35.68, 27.53, 27.53, 27.53; HRMS (ESI-TOF): Calcd m/z: [M+H]⁺Calcd for C₁₉H₂₄N₅O₆ 418.1721; Found 418.1716; [M+Na]⁺ Calcd for C₁₉H₂₃N₅O₆Na; 440.1541; Found 440.1536.

### Example 36: Synthesis of 3-(2-(allyloxy) ethyl)-6-amino-1,3,5-triazine-2,4(1H,3H)-dione (17a)

A solution of 16a in 50% TFA in DCM was stirred for 45 min at 0-4° C. The reaction mixture was concentrated in vacuo; the resulting residue was dissolved in diethyl ether and the solid was filtered in vacuum to afford 17a as a white solid.

Yield: 80%; mp. >250; The ¹H NMR (500 MHz, CDCl₃) δ 11.08 (bs., 1 H), 7.69 (bs., 1 H), 6.47 (bs., 1 H), 5.84 (tdd, J=5.3, 10.4, 17.2 Hz, 1 H), 5.23-5.22 (m, 2 H), 5.10 (m, 2 H), 3.92 (td, J=1.5, 5.3 Hz, 2 H), 3.83 (t, J=6.5 Hz, 2 H), 3.48 (t, J=6.3 Hz, 2 H) ¹³C NMR (125 MHz, CDCl₃) δ 156.16, 156.16, 135.13, 116.32, 70.63, 69.32, 66.20, 41.01; HRMS (ESI-TOF): Calcd m/z: [M+H]⁺ Calcd for C₈H₁₃N₄O₃⁺ 213.0982; Found 213.0982.

### Example 37: Synthesis of 2-(4-amino-2,6-dioxo-3,6-dihydro-1,3,5-triazin-1(2H)-yl) ethyl acrylate (17b)

The synthetic method of 17a was adopted to synthesize 17b.

Yield: 78%; mp.>300° C.; ¹H NMR (400 MHz, DMSO-d⁶) δ 7.14 (bs., 1 H), 6.30-6.26 (dd, J=1.6, 17.3 Hz, 1 H), 6.13-6.06 (dd, J=10.4, 17.3 Hz, 1 H), 5.90-5.93 (dd, J=1.5, 10.4 Hz, 1 H), 4.27-4.24 (t, J=5.4 Hz, 2 H), 3.97-3.94 (t, J=5.5 Hz, 2 H); ¹³C NMR (100 MHZ, DMSO-d⁶) δ 165.55, 156.20, 153.06, 150.26, 131.79, 128.40, 61.46, 40.35. HRMS (ESI-TOF): Calcd m/z: [M+H]⁺ Calcd for C₈H₁₁N₄O₄⁺ 227.0775; Found 227.0775.

### Example 38: Synthesis of N-(2-(4-amino-2,6-dioxo-3,6-dihydro-1,3,5-triazin-1(2H)-yl) ethyl) acrylamide (17c)

The synthetic method of 16a was adopted to synthesize 17c.

Yield: 78%; mp.>300° C.; Note: no solubility in DMSO-d⁶, HRMS (ESI-TOF): Calcd m/z: [M+H]⁺ Calcd for C₈H₁₂N₅O₃⁺ 226.0935; Found 227.0935.

### Example 39: Synthesis of 2-(2-(4-amino-2,6-dioxo-3,6-dihydro-1,3,5-triazin-1(2H)-yl) ethyl)-3a, 4,7,7a-tetra hydro-1H-4,7-methanoisoindole-1,3(2H)-dione (17d)

The synthetic method of 16a was adopted to synthesize 17d.

Yield: 84%; mp.>250° C.; ¹H NMR (400 MHz, DMSO-d⁶) δ 7.11 (bs., 3 H), 6.01 (s, 2 H), 3.76-3.73 (m, 2 H), 3.46-3.44 (d, J=10.7 Hz, 2 H), 3.24-320 (d, J=13.0 Hz, 4 H), 1.54 (s, 2 H); ¹³C NMR (101 MHz, DMSO-d⁶) δ 177.87, 177.55, 168.43, 156.48, 156.48, 153.47, 134.86, 134.86, 52.31, 45.82, 44.60, 39.41, 39.41, 36.19; HRMS (ESI-TOF): Calcd m/z: [M+H]⁺ Calcd for C₁₄H₁₆N₅O₄⁺ 318.1197; Found 318.1197.

### Advantages of the Present Invention

The novel triazine-based Janus G-C nucleobases of general formula (I) as potential building blocks for multiple application purposes.
The Janus G-C base, endowed with self-complementary H-bonding codes reminiscent of guanine (G) and cytosine (C) nucleic acid bases, is capable of undergoing efficient self-assembly leading to supramolecular polymers.
The orthogonally protected PNA building blocks carrying the triazine-based Janus G-C nucleobase of general formula (I) find application in developing PNAs useful for DNA/RNA recognition studies.
The novel class of polymer building blocks (16) carrying multi-facial H-bonding sites have potential utility in the development of smart/self-healing/functional polymers.

1. A triazine based self-assembling Janus G-C-base motif of formula (I); 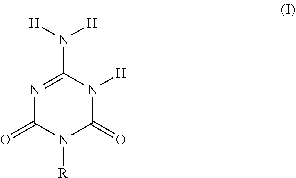
wherein, ‘R’ is selected from the group consisting of linear or branched unsubstituted and substituted C1-C7 alkyl, unsubstituted and substituted aryl, unsubstituted and substituted natural amino acids, protected unsubstituted and substituted natural amino acids, linear or branched unsubstituted and substituted C1-C7 alcohols, and linear or branched unsubstituted and substituted C1-C7 amines.

2. The triazine based self-assembling Janus G-C-base motif of formula (I) as claimed in claim 1, wherein the motif of formula (I) is selected from the group consisting of: 6-amino-3-benzyl-1,3,5-triazine-2,4 (1H,3H)-dione (Ia); 6-amino-3-neopentyl-1,3,5-triazine-2,4 (1H,3H)-dione (Ib); 6-amino-3-(2-hydroxyethyl)-1,3,5-triazine-2,4(1H,3H)-dione (Ic); 6-amino-3-(2-aminoethyl)-1,3,5-triazine-2,4(1H,3H)-dione (Id); Benzyl 2-(4-amino-2,6-dioxo-3,6-dihydro-1,3,5-triazin-1(2H)-yl) acetate (Ie); Benzyl 2-(4-amino-2,6-dioxo-3,6-dihydro-1,3,5-triazin-1(2H)-yl)-6-(((benzyloxy) carbonyl) amino) hexanoate (If); and Benzyl (S)-2-(4-amino-2,6-dioxo-3,6-dihydro-1,3,5-triazin-1(2H)-yl)-5-guanidino pentanoate (Ig).

3. A process comprising:
i. reacting guanidine (1) with tert-butlyoxycarbonyl (BOC) in presence of NaOH base, acetone and water at a temperature in a range of 25-30° C. for a period in a range of 9-10 hours to obtain Boc-protected guanidine (2);
ii. reacting the Boc-protected guanidine (2) obtained in step i) with carbonyldiimidazole (CDI) in dichloromethane as a solvent at a temperature in a range of 25-30° C. for a period in a range of 5-6 hours to obtain imidazole carbonyl-coupled reactive intermediate (3);
iii. reacting the intermediate (3) obtained in step ii) with R—NH₂ (II) in presence of a base and acetonitrile as a solvent at a temperature in a range of 45-50° C. for a period in a range of 4-5 hours to obtain amidino urea (4); wherein, ‘R’ is selected from the group consisiting of linear or branched unsubstituted and substituted C1-C7 alkyl, unsubstituted and substituted aryl, unsubstituted and substituted natural amino acids, protected unsubstituted and substituted natural amino acids, linear or branched unsubstituted and substituted C1-C7 alcohols, and linear or branched unsubstituted and substituted C1-C7 amines;
iv. cyclizing the amidino urea (4) obtained in step iii) with carbonyldiimidazole (CDI) at a temperature in a range of 30-35° C. for a period in a range of 45-48 hours to obtain the Boc protected Janus G-C nucleobase (5); and
v. deprotecting the nucleobase (5) obtained in step iv) with a deprotecting agent at a temperature in a range of 0-4° C. for a period in a range of 40-45 minutes to obtain a triazine based self-assembling Janus G-C-base motif of formula (I);
wherein the deprotecting agent in step (v) is selected from H₂/Pd-C for the benzyl protecting group, and 95% TFA in DCM for the Boc and the Pbf protecting group;

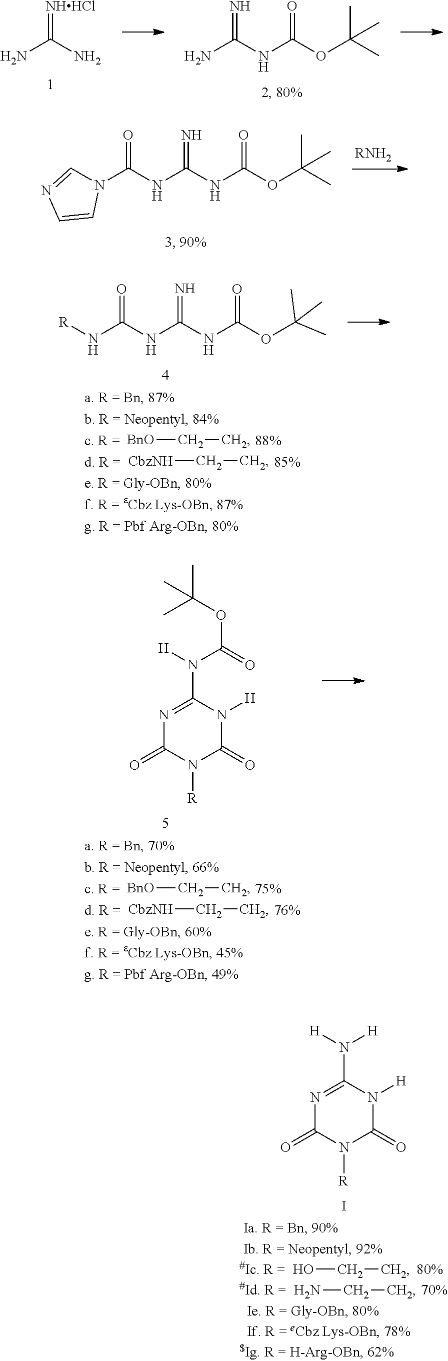

4.-13. (canceled)

14. The process as claimed in claim 3, wherein the compound of formula (Id) is crystallized from hot aqueous methanol containing traces of hydrochloric acid (HCl).

15. The process as claimed in claim 3, wherein the base in step iii) is selected from the group consisting of organic base, inorganic base or combination thereof; wherein the organic base is selected from a group consisting of ethylamine, triethylamine, DIPEA, pyridine; wherein the inorganic base is selected from the group consisting of sodium hydroxide, alkali or alkaline earth metal carbonates and bicarbonates.

16. The process as claimed in claim 3, further comprising:
i. converting Cbz-N-ethylenediamine (7) to Cbz-protected ethyl (2-aminoethyl) glycinate (Cbz-AEG-OEt) (8) in presence of a solvent and a base at a temperature in a range of 20-25° C. for a period in a range of 5-6 hours.
ii. reacting N-Boc ethylene diamine (9) with benzyl bromoacetate in presence of a solvent and a base at a temperature in a range of 30-35° C. for a period in a range of 5-6 hours to obtain Boc-protected benzyl ester (10);
iii. deprotecting Boc-protected benzyl ester (10) in TFA-DCM (1:1) at 0-4° C. for 40-45 minute, further reacting with Fmoc-OSu in presence of a base and a solvent at a temperature in a range of 30-35° C. for a period in a range of 1-2 hours to obtain Fmoc-protected benzyl-2-glycinate (Fmoc-AEG-OBn) (11).
iv. debenzylating glycine derived (5e) and/or arginine derived (5g) Boc-protected triazine benzyl esters to carboxylic acid (12a, b) by stirring at a temperature in a range of 30-35° C. under H₂/Pd-C atm for a period in a range of 5-6 hours;
v. coupling the intermediate (8) or (11) of step (I) and (III) at a temperature in a range of 30-35° C. for a period in a range of 12-16 hours with the compound (12a, b) to obtain Fmoc analog (13a, b) and Cbz analog (14a, b), respectively;

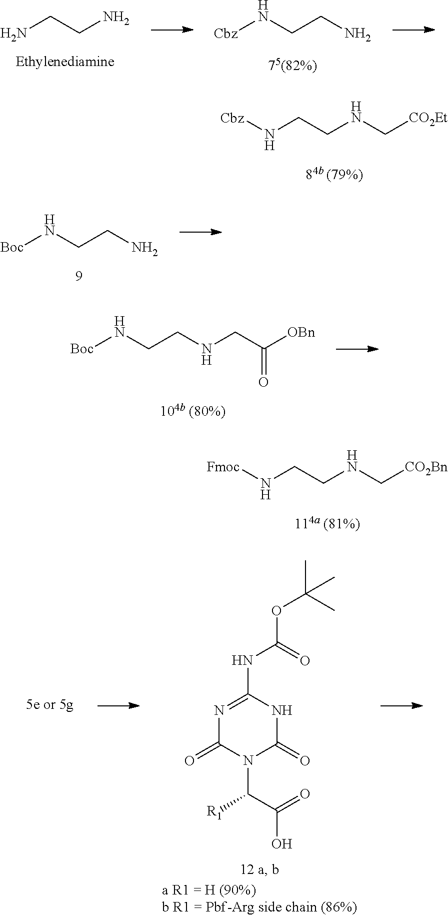

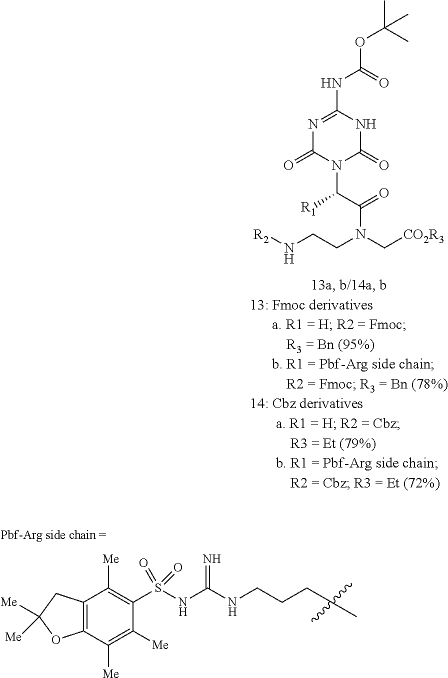

17. The process as claimed in claim 16, wherein the process is applicable in Fluorenylmethyloxycarbonyl (Fmoc) solid-phase synthesis or Carbobenzyloxy (Cbz) solution-phase synthesis of PNAs.

18. The process as claimed in claim 3, further comprising:
(a) reacting the imidazole carbonyl-coupled reactive intermediate (3) with amine of formula R₁—NH₂ (III) at a temperature in a range of 45-50° C. for a period in a range of 4-5 hours to obtain amidino urea (15); wherein R₁ is selected from the group consisting of below compounds of formulae a, b, c and d; 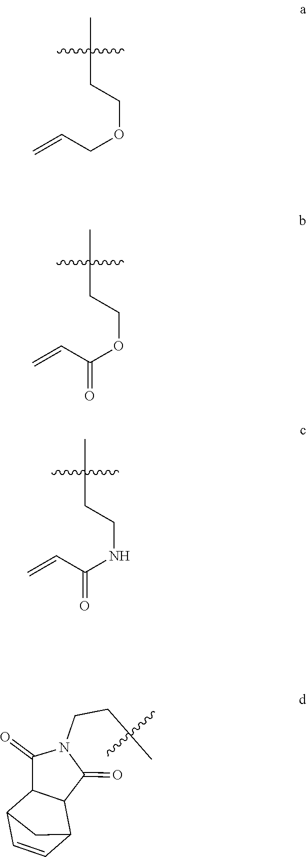
(b) cyclizing the amidino urea (15) obtained in step a) with carbonyldiimidazole (CDI) at a temperature in a range of 30-35° C. for a period in a range of 45-48 hours to obtain polymer building block (16);
(c) deprotecting the compound (16) at a temperature in a range of 0-4° C. for a period in a range of 40-45 minutes to obtain free amino triazine (17);

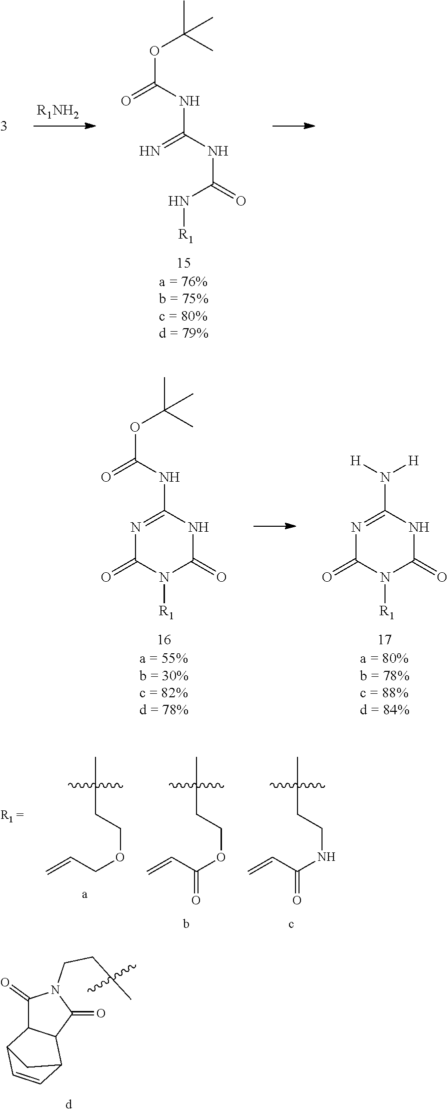

19. The process as claimed in claim 18, wherein the compounds of formula (17) are selected from the group consisting of: 3-(2-(allyloxy) ethyl)-6-amino-1,3,5-triazine-2,4(1H,3H)-dione (17a); 2-(4-amino-2,6-dioxo-3,6-dihydro-1,3,5-triazin-1(2H)-yl)ethyl acrylate (17b); N-(2-(4-amino-2,6-dioxo-3,6-dihydro-1,3,5-triazin-1(2H)-yl) ethyl) acrylamide (17c); and 2-(2-(4-amino-2,6-dioxo-3,6-dihydro-1,3,5-triazin-1(2H)-yl)ethyl)-3a, 4,7,7a-tetra hydro-1H-4,7-methanoisoindole-1,3(2H)-dione(17d).

20. The process as claimed in claim 18, wherein the compounds of formula (16) are subjected to covalent polymerization.

21. The process as claimed in claim 19, wherein the compound of formula (17b) is crystallized from hot dimethylsulfoxide (DMSO).
